SEQUENCE LISTING (7 sequences)

SEQ ID NO: 1 - DNA, 20 bp, Artificial Sequence
  Synthetic oligonucleotide; Barcode adapter, double stranded 1st strand
gatcggaagagcacacgtct

SEQ ID NO: 2 - DNA, 33 bp, Artificial Sequence
  Synthetic oligonucleotide; barcode adapter, double stranded 2nd strand
acactctttccctacacgacgctcttccgatct

SEQ ID NO: 3 - DNA, 58 bp, Artificial Sequence
  Synthetic oligonucleotide; PE 1.0
  modified_base (57)..(57): phosphorothioate bond
aatgatacggcgaccaccgagatctacactctttccctacacgacgctcttccgatct

SEQ ID NO: 4 - DNA, 66 bp, Artificial Sequence
  Synthetic oligonucleotide; index primer
  misc_feature (25)..(32): n is a, c, g, or t
  modified_base (65)..(65): phosphorothioate bond
caagcagaagacggcatacgagatnnnnnnnngtgactggagttcagacgtgtgctcttccgatct

SEQ ID NO: 5 - DNA, 18 bp, Artificial Sequence
  Synthetic oligonucleotide; 16S forward primer
actcctacgggaggcagc

SEQ ID NO: 6 - DNA, 19 bp, Artificial Sequence
  Synthetic oligonucleotide; 16S reverse primer
ttaccgcggctgctggcac

SEQ ID NO: 7 - DNA, 219 bp, Artificial Sequence
  Synthetic oligonucleotide; gBLOCK
ggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgag

(19) United States
(12) Patent Application Publication
(10) Pub. No.: US 2023/0034247 A1
(43) Pub. Date: Feb. 2, 2023

(54) METHOD AND COMPOSITION FOR TREATING OR DECREASING GUT MICROBIOME DYSBIOSIS INDUCED BY A PRIOR ANTIBIOTIC TREATMENT

(71) Applicant: Agency for Science, Technology and Research, Singapore (SG)

(72) Inventors: Kern Rei CHNG, Singapore (SG); Aarthi RAVIKRISHNAN, Singapore (SG); Niranjan NAGARAJAN, Singapore (SG)

(73) Assignee: Agency for Science, Technology and Research, Singapore (SG)

(21) Appl. No.: 17/367,837

(22) Filed: Jul. 6, 2021

(51) Int. Cl. A61K 35/741; C12Q 1/04; A61P 1/00

(52) U.S. Cl. CPC A61K 35/741; C12Q 1/04; A61P 1/00; A61K 2035/115

(57) ABSTRACT

The invention relates to using a class of microbial species that can contribute to robust recovery of the microbiome after antibiotic usage. In particular, the inventors of this invention have identified 21 bacterial species exhibiting robust association with ecological recovery post antibiotic therapy. As such, in an aspect of the invention, there is provided a use of composition comprising at least one of or any combination of microorganisms selected from the group consisting of: Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile for treating or decreasing gut microbiome dysbiosis induced by a prior antibiotic treatment.

A sequence listing as presented in the ASCII text file named 33D7295.txt, created on Sep. 30, 2021 and having a file size of 2,455 bytes, is herein incorporated by reference in its entirety.

The invention relates to using a class of microbial species that can contribute to robust recovery of the microbiome after antibiotic usage.

The human gut microbiome harbors trillions of bacteria providing diverse metabolic capabilities and with essential roles in host health, particularly energy metabolism, immune homeostasis, and xenobiotic metabolism. A stable consortium of commensal microbiota is also believed to play a key role in resisting colonization by pathogens, with reduced diversity being associated with increased risk for infections. Several recent studies have further highlighted the importance of the gut microbiome for host health, particularly in infants and the elderly, with alterations and loss of diversity being associated with various metabolic, immunological and neurological diseases, and poorer response to cancer immunotherapy.

Among the many factors that are known to perturb the gut microbiome, antibiotics are the major cause of profound and long-term alterations. Antibiotics are widely used in farming and healthcare, and global consumption is estimated to have increased by 65% from the years 2000 to 2015. While the impact of antibiotics on host health through microbiome disruption is likely to be significant, it has not been fully quantified to date. Antibiotic associated diarrhea and Clostridium difficile colitis are common early complications of microbiome disruption, while antibiotics also select for drug resistance genes and organisms, thus creating a reservoir for transmission of resistance cassettes. In the medium to long term, recovery of the microbial community can be slow and variable, and is conditioned on the initial state. Epidemiological and model organism studies suggest that long-term consequences of antibiotic usage include immunological diseases in children, metabolic diseases in adults, and an increased risk of infections.

Despite mounting evidence on the importance of gut microbiome function and how antibiotic usage can severely impact it, understanding of the post-antibiotic recovery process is limited. Several studies have noted that high initial diversity in the gut microbiome may be associated with better recovery from antibiotic-induced perturbations. In addition, the carriage of specific antibiotic resistance genes has been linked with the recovery process in some studies. While it is expected that bacteria that are resistant to the antibiotic used will have an advantage in seeding the repopulation of the gut, it is unclear if antibiotic resistance alone is sufficient or necessary to recover the ecological and functional richness of the gut microbiome. In particular, it is not known as to which specific groups of microbial taxa, and the functions they perform, accelerate or impede the process and explain the substantial variability in speed and extent of recovery that is seen across individuals. For example, while commonly used probiotics can be generally beneficial to host health, their utility after antibiotic treatment remains unclear, with a recent study providing evidence that they may in fact delay microbiome recovery.

The interactions between species play a key role in the recovery of many ecosystems after severe perturbations. Typically, reseeding by a few keystone species is essential to trigger a chain of food-web interactions that eventually lead to recovery of the overall ecosystem. Several important constituents of the healthy gut microbiome have been identified (e.g. Bacteroides species) and correlations in their abundance have been used to postulate cross-feeding interactions. However, the role of these species and their interactions in the context of post-antibiotic microbiome recovery have not been explored.

The listing or discussion of an apparently prior-published document in this specification should not necessarily be taken as an acknowledgement that the document is part of the state of the art or is common general knowledge.

Any document referred to herein is hereby incorporated by reference in its entirety.

Here, a metagenome-wide association approach has been employed to identify microbial species and functions that could contribute to robust recovery of the microbiome after antibiotic usage. It is then shown how in vivo human metagenomic data from multiple cohorts supports a mechanistic model where gut microbiome recovery is facilitated by carbohydrate degradation and microbial cross-feeding triggered by a subset of the identified species. Validation experiments in a mouse model demonstrate how recovery-associated bacterial species (RABs) can synergistically provide a >100-fold boost to absolute microbial abundance and higher diversity in the gut microbiome after antibiotic treatment. Systematic investigations using higher-order combinations of RABs can thus help the understanding of the interactions between them that likely contribute to the complex ecological processes underlying gut microbiome recovery.

Advantageously, these microbial species contain specific enzymes that help degrade a wide range of host- and diet-derived carbohydrates, thereby serving as primary producers in the gut to provide food and energy for other bacteria that cannot break down the carbohydrates. This helps rebuild the food web in the gut, eventually boosting the recovery of a diverse, healthy microbial community.

In an aspect of the invention, there is provided a method of treating or decreasing gut microbiome dysbiosis induced by a prior antibiotic treatment, the method comprising administering to a subject an effective amount of a composition comprising at least one of or any combination of microorganisms selected from the group consisting of: Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

By “treating or decreasing gut microbiome dysbiosis”, it is meant to also include “reverting” or “reversing” the effects of a prior antibiotic treatment has caused to the gut microbiota in a subject. It also includes “reverting”, “reversing” (and then maintaining) a microbiota diversity in a healthy subject. As such, these phrases are meant to express that species diversity (species richness and/or species evenness) of the microbiota of an individual will not be significantly modified or affected, especially in case of dysbiosis. In particular, maintaining the microbiota diversity could help the subject to recover faster in case of risk of dysbiosis or could avoid the dysbiosis to worse. The phrases “increase of microbiota diversity”, “promote recovery of microbiota diversity”, “treatment/decrease/reduction/of dysbiosis” etc. may be used to express an increase in species diversity (species richness and/or species evenness) of the microbiota of an individual. Methods for the calculation of species diversity, species richness and species evenness are known in the art and include but are not limited to Simpson's Index, Simpson's Index of Diversity and Simpson's Reciprocal Index, Chao Index and Shannon Index.

In addition, the above phrases also are intended to include “accelerate the increase of the intestinal microbiota diversity”, “promote recovery of the intestinal microbiota diversity”, “favour the return to a baseline/normal/healthy intestinal microbiota diversity”, “accelerate the decrease/reduction/disappearance of the dysbiosis” etc. may be used to express that the diversity (richness and/or evenness) of the microbiota of individuals having an intestinal dysbiosis after a treatment by antibiotics increases statistically more rapidly in subjects who take the probiotic strain than in control subjects who do not, so that the structure of the microbiota three weeks after the antibiotic treatment is statistically closer to the structure before said treatment in subjects who take the probiotic strain than in control subjects who do not.

By “dysbiosis”, it is meant to a change in microbiota commensal species diversity as compared to a healthy or general population and shall include decrease of beneficial microorganisms and/or increase of pathobionts (pathogenic or potentially pathogenic microorganisms) and/or decrease of overall microbiota species diversity. Many factors can harm the beneficial members of the intestinal microbiota leading to dysbiosis, including antibiotic use, psychological and physical stress, radiation, and dietary changes.

By “microorganisms”, it is meant include any bacterial strain or species shall be taken to include bacteria derived therefrom wherein said bacteria retain the capacity to decrease intestinal dysbiosis of a subject, preferably a subject having an antibiotic-induced dysbiosis. Strains derived from a parent strain which can be used according to the present invention include mutant strains and genetically transformed strains. These mutants or genetically transformed strains can be strains wherein one or more endogenous gene(s) of the parent strain has (have) been mutated, for instance to modify some of their metabolic properties (e.g., their ability to ferment sugars, their resistance to acidity, their survival to transport in the gastrointestinal tract, their post-acidification properties or their metabolite production). They can also be strains resulting from the genetic transformation of the parent strain to add one or more gene(s) of interest, for instance in order to give to said genetically transformed strains additional physiological features, or to allow them to express proteins of therapeutic or vaccinal interest that one wishes to administer through said strains. These mutants or genetically transformed strains can be obtained from the parent strain by means of conventional techniques for random or site-directed mutagenesis and genetic transformation of bacteria, or by means of the technique known as “genome shuffling”. Strains, mutants and variants derived from a parent species or strain and retaining the ability to maintain or increase intestinal microbiota diversity of a subject having an antibiotics-induced dysbiosis may be considered as being encompassed by reference to said parent species or strain as those 21 microorganisms recited in claim 1 of this application.

In various embodiments, the method comprises administering to a subject an effective amount of a composition comprising all 21 microorganisms: Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

In alternative embodiments, the method comprises administering to a subject an effective amount of a composition comprising either one or both of Bacteroides thetaiotaomicron and Bifidobacterium adolescentis.

By “composition”, it is meant to include any “synthetic composition” or formulation that is artificially made and not naturally occurring. Any such suitable formulation would include any process of isolating, purifying and manufacture to ensure said formulation is safe for human consumption.

In another aspect of the invention, there is provided a synthetic composition for treating or decreasing gut microbiome dysbiosis induced by a prior antibiotic treatment, the composition comprising at least one of, or a combination of, a microorganisms selected from the group consisting of, or consisting essentially of: Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

For example, the synthetic composition may be a probiotic or a pharmaceutical formulation.

In various embodiments, the composition comprises essentially of all 21 microorganisms: Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

In various embodiments, the composition comprises either one of, or both Bacteroides thetaiotaomicron and Bifidobacterium adolescentis.

In various embodiments, the composition is a probiotic, food product or a pharmaceutical composition.

The term “probiotic”, “food product” and “pharmaceutical composition” have generally accepted definitions. For example, probiotics may be defined as live microorganisms thought to be healthy for the host organism; digestive enzymes may be defined as enzymes that break down polymeric macromolecules into their smaller building blocks in order to facilitate their absorption by the body; dietary supplements may be defined as a preparation intended to supplement the diet and provide nutrients that may be missing or may not be consumed in sufficient quantities in a human's diet.

The selected microorganisms of the invention may be in a liquid culture or dried form for administration. The drying of bacterial strains after production by fermentation is known to the skilled person. See for example, EP 0 818 529 (SOCIETE DES PRODUITS NESTLE), which is incorporated by reference in its entirety, where a drying process of pulverization is described. In some embodiments, the microorganisms may be lyophilized, pulverized and powdered. Usually, bacterial microorganisms are concentrated from a medium and dried by spray drying, fluidised bed drying, lyophilisation (freeze drying) or other drying process. Microorganisms can be mixed, for example, with a carrier material such as a carbohydrate such as sucrose, lactose or maltodextrin, a lipid or a protein, for example milk powder during or before the drying.

The bacterial strain need not necessarily be present in a dried form. It may also be suitable to mix the bacteria directly after fermentation with a food product and, optionally, perform a drying process thereafter. Such an approach is disclosed in PCT/EP02/01504, which is incorporated by reference in its entirety. Likewise, a probiotic composition of the invention may also be consumed directly after fermentation. Further processing, for example, for the sake of the manufacture of convenient food products, is not a precondition for the beneficial properties of the bacterial strains provided in the probiotic composition.

The compositions according to the present invention may be enterally consumed in any form. They may be added to a nutritional composition, such as a food product. On the other hand, they may also be consumed directly, for example in a dried form or directly after production of the biomass by fermentation.

According to the subject invention, the bacterial strain(s) can be provided in an encapsulated form in order to ensure a high survival rate of the micro-organisms during passage through the gastrointestinal tract or during storage or shelf life of the product.

The compositions of the subject invention may, for example, be provided as a probiotic composition that is consumed in the form of a fermented, dairy product, such as a chilled dairy product, a yogurt, or a fresh cheese. In these later cases, the bacterial strain(s) may be used directly also to produce the fermented product itself and has therefore at least a double function: the probiotic functions within the context of the present invention and the function of fermenting a substrate such as milk to produce a yogurt.

If the bacterial strain is added to a nutritional formula, the skilled person is aware of the possibilities to achieve this. Dried, for example, spray dried bacteria, such as obtainable by the process disclosed in EP 0 818 529 (which is incorporated herein by reference in its entirety) may be added directly to a nutritional formula in powdered form or to any other food product. For example, a powdered preparation of the bacterial strain(s) of the invention may be added to a nutritional formula, breakfast cereals, salads, a slice of bread prior to consumption.

In various embodiments, the microorganism composition is a liquid culture that may be administered to a subject.

Bacterial strain(s) of the invention may be added to a liquid product, for example, a beverage or a drink. If it is intended to consume the bacteria in an actively-growing state, the liquid product comprising the bacterial strain(s) should be consumed relatively quickly upon addition of the bacteria. However, if the bacteria are added to a shelf-stable product, quick consumption may not be necessary, so long as the bacterial strain(s) are stable in the beverage or the drink.

WO 98/10666, which is incorporated herein by reference in its entirety, discloses a process of drying a food composition and a culture of probiotic bacteria conjointly. Accordingly, the subject bacterial strain(s) may be dried at the same time with juices, milk-based products or vegetable milks, for example, yielding a dried product already comprising probiotics. This product may later be reconstituted with an aqueous liquid.

By “food product”, it is also meant to include any food supplements made from compounds usually used in foodstuffs, but which is in the form of tablets, powder, capsules, potion or any other form usually not associated with aliments, and which has beneficial effects for one's health. It is meant to also include any “functional food” which has beneficial effects for one's health in addition to providing nutrients. In particular, food supplements and functional food can have a physiological effect—for the prophylaxis, amelioration or treatment of a disease, for example a chronic disease.

The composition can be a pharmaceutical composition or a nutritional composition. In various embodiments, the composition is a nutritional composition such as a food product (including a functional food) or a food supplement.

Nutritional compositions which can be used according to the invention include dairy compositions, preferably fermented dairy compositions. The fermented compositions can be in the form of a liquid or in the form of a dry powder obtained by drying the fermented liquid. Examples of dairy compositions include fermented milk and/or fermented whey in set, stirred or drinkable form, cheese and yoghurt. The fermented product can also be a fermented vegetable, such as fermented soy, cereals and/or fruits in set, stirred or drinkable forms.

Nutritional compositions which can be used according to the invention also include baby foods, infant milk formulas and infant follow-on formulas. In various embodiments, the fermented product is a fresh product. A fresh product, which has not undergone severe heat treatment steps, has the advantage that the bacterial strains present are in the living form.

In various embodiments, the pharmaceutical composition is formulated for oral administration. The pharmaceutical composition may comprise a coating, optionally wherein the coating is an enteric coating. The coating material comprises at least one of a saccharide, a polysaccharide, and a glycoprotein extracted from at least one of a plant, a fungus, and a microbe, optionally wherein the at least one of a saccharide, a polysaccharide, and a glycoprotein includes one or more of corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum.

In various embodiments, the pharmaceutical composition is formulated with a germinant.

The probiotic ingredients of the composition may be present in an effective dose. For example, at the time of manufacture, the probiotic ingredients may total at least 6×10⁹ colony forming units (cfu) and may include at least 13×10⁹ cfu of probiotics or more. In various embodiments, the probiotic ingredients total at least 13×10⁹ cfu of probiotics. In various embodiments, the probiotic ingredients total at least 14×10⁹ cfu of probiotics. A colony forming unit (cfu) is generally accepted as a measure of viable bacterial or fungal numbers. Such quantity of probiotic ingredient may facilitate providing a consumer with an effective dose of probiotics at the time of ingestion, as the inventor has realized that probiotics may be destroyed during storage due to undesirable environments (e.g., temperature extremes) and other reasons. In various embodiments, the composition is formulated in a dosage form at least about 1×10⁴ colony forming units of bacteria.

In various embodiments, the consumption or administration of a dose of between about 10⁸ and about 10¹¹ colony forming unit (CFU) of at least one of or any combination of microorganisms selected from the group consisting of: Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile. In other embodiments, it could be between about 10⁸ and about 10⁹. Alternatively, it could be between about 10⁹ and about 10¹⁰ colony forming unit (CFU) and in an alternative embodiment between about 10¹⁰ and about 10¹¹ colony forming unit (CFU). In various embodiments least 1, 2, 3, or 4 doses are provided within a 24 hour time period. It is further preferred that the daily dosage regimen is maintained for at least about 1, 2, 3, 4, 5, 6 or 7 days, or in alternative embodiment for at least about 1, 2, 3, 4, 5, 6 or 7 weeks.

The composition of the invention may be incorporated into a food product, e.g. yoghurt. Alternatively, to facilitate protection of the composition, capsules comprising the composition may be and are preferably stored in blister packs. That is, the blister packs may seal the capsule from a surrounding environment and thus, extend the life of the effective ingredients of the composition.

Oral delivery of the composition is accomplished via a 2 to 4 ounce emulsion or paste mixed with an easy to eat food such as a milk shake or yoghurt. The microencapsulated bacterial probiotic and prebiotic can be administered along with the mixture of sorbents in the emulsion or paste or separately in a swallowable gelatin capsule.

A mathematical model of solute transport of oral sorbents has been developed based on the diffusion controlled solute flux into the intestinal lumen followed by physical binding or chemical trapping (Gotch et al. Journal of Dialysis 1976-1977 1(2): 105-144). This model provides the theoretical basis of solute removal through the gut.

Any method of using the composition may be used as desired by consumers of the composition. A particularly advantageous program may be to take a single capsule of the composition on a daily basis until the effects of the gut microbiome dysbiosis is reduced or eliminated.

In another aspect of the invention, there is provided a use of a composition according to any one aspect of the invention in the manufacture of a medicament for treating or decreasing gut microbiome dysbiosis induced by antibiotic treating that had received an antibiotic treatment.

In yet another aspect of the invention, there is provided a method for predicting the likelihood of antibiotics-induced microbiome dysbiosis recovery in a subject, the method comprising: (a) determining a gut microbiome signature of the subject by determining an amount of, or presence or absence of, each microorganism in a group of microorganisms present in a sample obtained from the subject; and (b) applying a prediction model to assess the gut microbiome signature with respect to a gut profile representative of good gut health to obtain a likelihood of antibiotics-induced microbiome dysbiosis recovery in the subject, wherein the group of microorganisms comprises Alistipes putredinis, Alistipes shahii, Bacteroides caccae, Bacteroides coprocola, Bacteroides eggerthii, Bacteroides intestinalis, Bacteroides stercoris, Bacteroides thetaiotaomicron, Bacteroides uniformis, Bifidobacterium adolescentis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile, and wherein the prediction model is trained using a dataset of microbiome profiles of a plurality of subjects who have recovered from antibiotics-induced microbiome dysbiosis, and a plurality of subject who have not recovered from antibiotics-induced microbiome dysbiosis.

In various embodiments, the method of the invention applies the prediction model to assess the gut microbiome signature with respect to a gut profile representative of good gut health to obtain a disparity level associated with the likelihood of antibiotics-induced microbiome dysbiosis recovery in the subject. Such a disparity level may be ascertained or derived based on any observation or score differences between a subject's gut microbiome signature and the signature of a good gut health signature. As will be described in detail below, the prediction model may be trained to determine what a good gut health signature may be (or develop a good gut health signature) based on the input of data provided by samples associated with the presence/absence/amounts of the 21 RABs of this invention in the samples. As such, by “likelihood”, it may refer to any quantifiable figure or form based on such a disparity level, e.g. a percentage disparity level may correlate to a percentage that determines whether a subject is less or more likely to recover.

As will be understood by those skilled in the art, such a prediction is usually not intended to be correct for 100% of the subjects to be assessed by the present invention. The method for predicting a subject's likelihood of recovery, however, requires that the prediction to be at the likelihood of recovery, or not, is correct for a statistically significant portion of the subjects (e.g. a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details may be found in Dowdy and Wearden, Statistics for Research, John Wiley & Sons, New York 1983. Examplary confidence intervals are at least 90%, at least 95%, at least 97%, at least 98% or at least 99%. The p-values may include 0.1, 0.05, 0.01, 0.005, or 0.0001.

By “recovery”, it is meant to refer to any reduction or relief in or of the effects and/or discomfort associated with gut microbiome dysbiosis a subject may have been experiencing.

The identification of the microorganisms, and their relationship and dependence on each other (e.g. metabolically) of the present invention have led to the use of the automatic or machine learning to create a machine learning model for predicting the likelihood of antibiotics-induced microbiome dysbiosis recovery in a subject.

In various embodiments, the prediction model comprises a machine learning probability model. The prediction model may comprise a random forest classification model, or a linear discriminant analysis model, or a sparse logistic regression model, or a conditional inference tree model. In various embodiments, one measure of dysbiosis may be arriving at a diversity score and based on Shannon entropy, i.e. for relative abundances pᵢ for species i, sum over all i of pᵢ log(pᵢ) may be computed. The prediction model may be any model including a decision tree.

In various embodiments, the gut microbiome signature of the subject is determined using a statistical analysis.

In various embodiments, the sample is a faecal sample obtained from the subject.

In other embodiments, the method of determining the likelihood of gut microbiome recovery post-antibiotic therapy, may include determining levels of carbohydrate-active enzyme (CAZyme) families from gut metagenomic data obtained from a sample, and/or determining the levels of the 21 RABs of the invention pre- and during antibiotic therapy.

In another aspect of the invention, there is provided a method for reducing antibiotics-induced gut microbiome dysbiosis in a subject, the method comprising: (a) determining a gut microbiome signature of the subject by determining an amount of, or presence or absence of, each microorganism in a group of microorganisms present in a sample obtained from the patient; and (b) administering to the subject a therapeutically effective amount of an agent which up-regulates at least one microbe which is down-regulated during a prior antibiotic treatment or administering to the subject a therapeutically effective amount of an agent which down-regulates a microbe which is up-regulated during a prior antibiotic treatment, thereby reducing antibiotics-induced gut microbiome perturbations in a subject, wherein the class of microbes comprises Alistipes putredinis, Alistipes shahii, Bacteroides caccae, Bacteroides coprocola, Bacteroides eggerthii, Bacteroides intestinalis, Bacteroides stercoris, Bacteroides thetaiotaomicron, Bacteroides uniformis, Bifidobacterium adolescentis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

In various embodiments, the agent is a probiotic and/or a prebiotic.

The probiotic is a bacterial population comprises Alistipes putredinis, Alistipes shahii, Bacteroides caccae, Bacteroides coprocola, Bacteroides eggerthii, Bacteroides intestinalis, Bacteroides stercoris, Bacteroides thetaiotaomicron, Bacteroides uniformis, Bifidobacterium adolescentis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

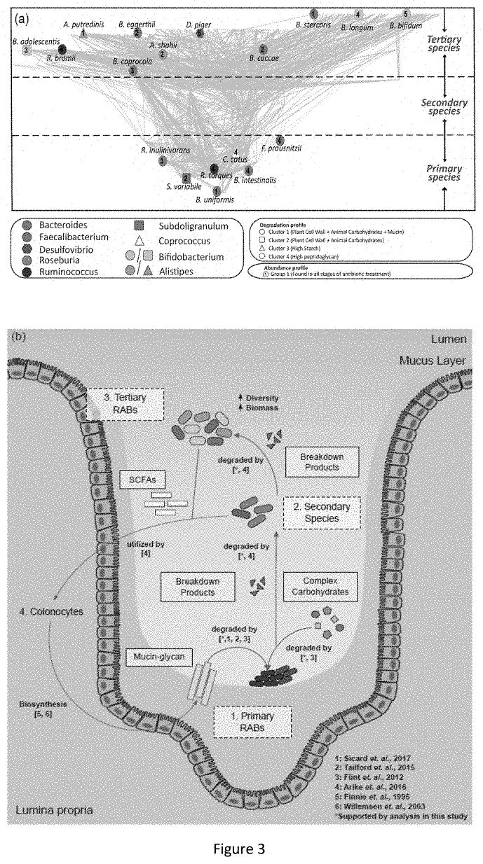

The choice of the agent for administration in step (b) will be dependent on the gut microbiome signature of the patient. This choice will be based on the relationship of the 21 RABs identified in the food web as will be explained below and shown in FIG. 3. By “food web”, it is meant to refer to that network of relationship between the 21 RABs realised by the present inventors. For example, Supplementary Data 7 (as described below) sets out the top interactions between the 21 RABs, e.g. Bifidobacterium adolescentis is the organism that benefits, and Subdoligranulum variabile is the organism that helps.

Prebiotics may include complex carbohydrates, amino acids, peptides, minerals, or other essential nutritional components for the survival of the bacterial composition. Prebiotics include, but are not limited to, amino acids, biotin, fructooligosaccharide, galactooligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carregenaan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) and xylooligosaccharides.

In yet another aspect of the invention, there is provided a method of determining the effect of a perturbation on a gut microbial community, the method comprising applying the perturbation to a cultured collection of a gut microbial community and determining the difference in the community before and after the application of the perturbation, wherein the difference in the cultured collection represents the effect of the perturbation on the original gut microbial community, wherein the gut microbial community comprises Alistipes putredinis, Alistipes shahii, Bacteroides caccae, Bacteroides coprocola, Bacteroides eggerthii, Bacteroides intestinalis, Bacteroides stercoris, Bacteroides thetaiotaomicron, Bacteroides uniformis, Bifidobacterium adolescentis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile. The perturbation is a diet related perturbation, an environmental perturbation, a genetic perturbation or a pharmaceutical perturbation.

In another aspect of the invention, there is provided a computer readable storage medium comprising computer readable instructions operable when executed by a computer to determine the likelihood of antibiotics-induced gut microbiome recovery in a subject, the computer readable instructions configured to perform a method of the invention.

In yet another aspect of the invention, there is provided an apparatus or system comprising: (a) a receiving unit configured to receive a dataset of values representing a gut microbiome signature of a subject by determining an amount of, or presence or absence of, each microorganism in a group of microorganisms present in a sample obtained from the subject; and (b) a processor configured to process a prediction model to assess the gut microbiome signature with respect to a gut profile representative of good gut health to obtain a likelihood of antibiotics-induced microbiome dysbiosis recovery in the subject, wherein the group of microorganisms comprises Bacteroides intestinalis, Bifidobacterium adolescentis, Alistipes putredinis, Alistipes shahii, Bacteroides caccae, Bacteroides coprocola, Bacteroides eggerthii, Bacteroides stercoris, Bacteroides thetaiotaomicron, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile, and wherein the prediction model may be trained using a dataset of microbiome profiles of a plurality of subjects who have recovered from antibiotics-induced microbiome dysbiosis, and a plurality of subjects who have not recovered from antibiotic-induced microbiome dysbiosis.

In various embodiments, the apparatus or system further comprises a memory for storing a prediction model, wherein the predictive model is configured to generate a prediction of the likelihood of recovery based upon an amount of, or presence or absence of, each microorganism in the group of microorganisms.

In order that the present invention may be fully understood and readily put into practical effect, there shall now be described by way of non-limitative examples only preferred embodiments of the present invention, the description being with reference to the accompanying illustrative figures.

In the Figures:

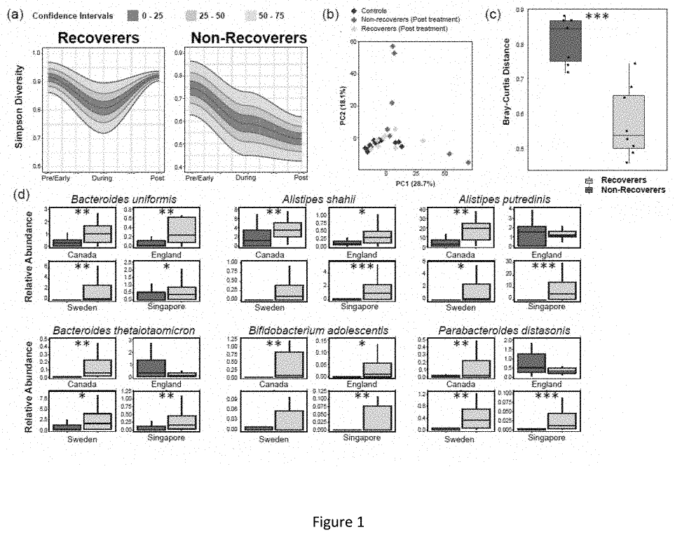

FIG. 1: Gut microbiome recovery profiles and key associated taxa. (a) Density plots showing the two different recovery profiles for microbial diversity (Simpson) that were observed in the antibiotic treatment cohorts (CA, SG). (b) Principal Component Analysis (PCA) plot showing the distribution of post-antibiotic gut microbiome profiles for recoverers and non-recoverers in relation to healthy control gut microbiome profiles (CA; n=8 for recoverers, n=7 for non-recoverers and n=18 for controls). (c) Boxplots showing the distribution of Bray Curtis Distances for post-antibiotic gut microbiomes for recoverers and non-recoverers in relation to healthy controls (median value; CA; n=8 for recoverers and n=7 for non-recoverers). ‘***’ represents p-value (one-sided Wilcoxon test) less than 0.001. (d) Relative abundance boxplots for 6 of the RABs that were identified in at least ¾ cohorts (Table 2) based on all timepoints. Note that ‘*’, ‘**’ and ‘***’ denote cohort-specific FDR adjusted p-values (one-sided Wilcoxon test; n=24 [CA], 32 [SW], 16 [EN] and 41 [SG] samples for recoverers and n=21 [CA], 24 [SW], 24 [EN] and 22 [SG] samples for non-recoverers) less than 0.05, 0.01 and 0.001 respectively. For all subfigures, boxplots are represented with center line: median; box limits: upper and lower quartiles; whiskers: 1.5×interquartile range; outlier points not included in visualization.

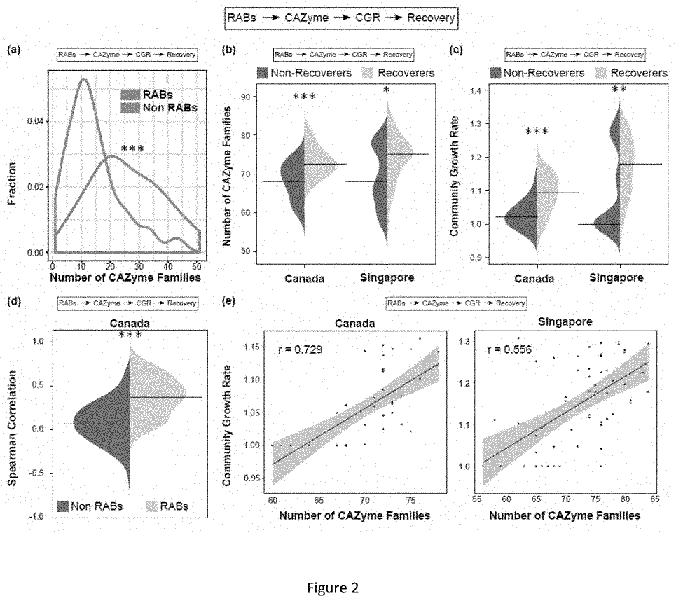

FIG. 2: Mechanistic model linking microbial functions with recovery. Subfigures provide evidence for a model of microbiome recovery based on RABs being enriched for carbohydrate degradation capabilities (CAZyme), which in turn promote faster community growth (CGR), and ultimately microbiome recovery (associations shown in each subfigure are highlighted in blue). (a) Empirical distributions for the number of CAZyme families in RABs and non-RABs showing that RABs are strongly enriched for CAZymes (two-sided Wilcoxon test). (b) Bean plots showing the variation in the number of CAZyme families (empirical distributions) detected in the gut microbiomes of recoverers and non-recoverers in the CA and SG cohorts (all timepoints; n=24 [CA], 41 [SG] samples for recoverers and n=21 [CA], 22 [SG] samples for non-recoverers). In both cohorts, recoverers have more CAZyme families represented in their metagenomes (one-sided Wilcoxon test). (c) Bean plots showing variation in the gut microbial community growth rate (empirical distributions) of recoverers and non-recoverers in the CA and SG cohorts (all timepoints; n=18 [CA], n=40 [SG] samples for recoverers and n=21 [CA], n=18 [SG] samples for non-recoverers). In both cohorts, recoverers have higher community growth rates (one-sided Wilcoxon test). (d) Bean plots showing that the abundance of RABs in the pre- and during phase of antibiotic treatment was better correlated (Spearman) to the post-treatment community growth rate of individuals in the CA cohort compared to non-RAB species (empirical distributions; one-sided Wilcoxon test; n=21 RABs, n=89 non-RABs; p-value>0.1 for SG cohort). (e) Correlation between the number of CAZyme families detected and the overall community growth rate across all gut microbiomes constituting the CA and SG cohorts (all timepoints). In both cohorts, community growth rates were consistently correlated with CAZyme diversity. Note that ‘*’, ‘**’ and ‘***’ denote p-values less than 0.05, 0.01 and 0.001 respectively for all subfigures. For all subfigures, bean plots are represented with beanline: median.

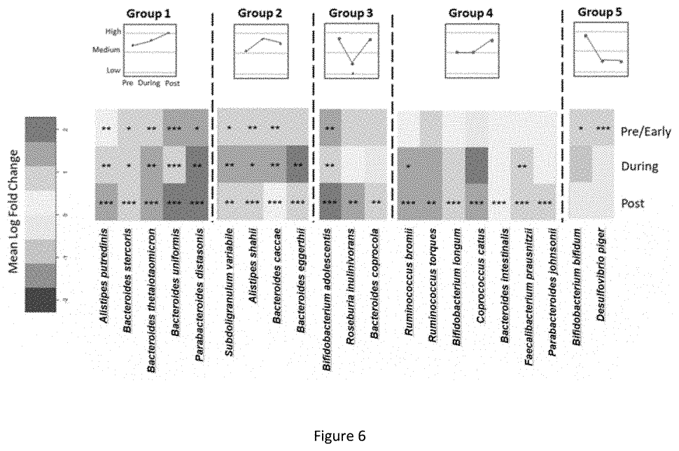

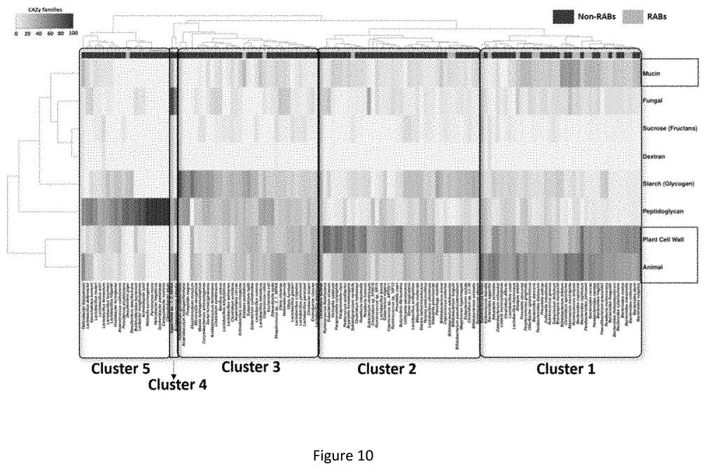

FIG. 3: Role of RABs in ecological recovery via the microbial food web. (a) Graph showing network structure of microbial dependencies inferred using an association rule mining approach, where an edge from species A to species B indicates that A's presence is required to have B in the community. Nodes are ordered from the bottom to the top such that species at the bottom have more outgoing edges than incoming edges (‘Primary Species’), while species at the top have more incoming edges than outgoing edges (‘Tertiary Species’). RABs (highlighted in different colors based on the genus they belong to) were observed either at the bottom or top of the graph. Many RABs at the bottom of the graph were from cluster 1 (degradation profile; FIG. 10), defined by mucin degrading CAZymes. Clusters based on abundance profile over time (FIG. 6) are indicated using numbers and do not seem to be biased in different regions of the graph. (b) Schematic representation of the gut showing a model for microbiome recovery based on these observations. RABs from cluster 1 (FIG. 10) colonize the epithelial mucosa better because of their mucin degrading capabilities (step 1), and since they can also break down dietary plant and animal derived carbohydrates (step 2), they act as primary species that facilitate the growth of other species (step 3). Some of the tertiary RABs and other species can produce short chain fatty acids (SCFAs), which are then utilized by colonocytes for their growth leading to increased mucin production (step 4). This positive feedback loop may enable faster ecological recovery in terms of diversity and biomass.

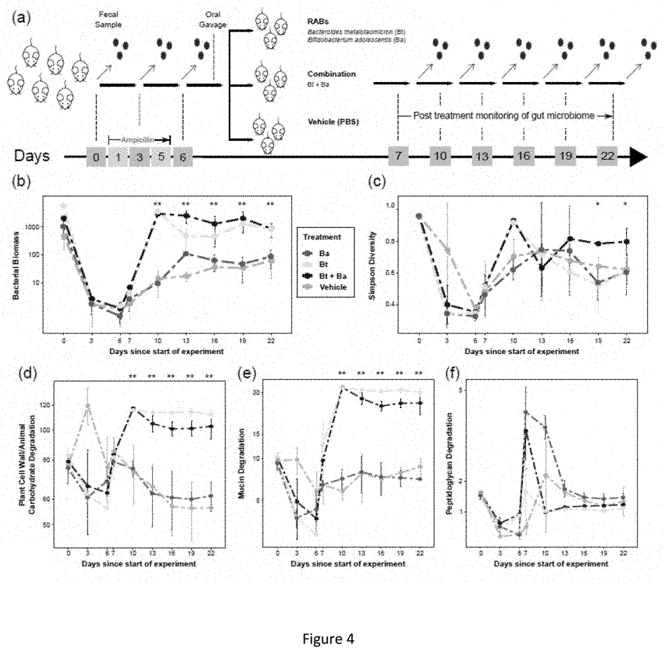

FIG. 4: Promoting microbiome recovery in a mouse model using RABs. (a) Schematic depicting the design of a mouse model experiment to study the impact of RABs in promoting microbiome recovery. Mice were given antibiotics for 5 days, followed by a rest day and gavage of different RABs and controls (Vehicle: n=5, Ba: n=6, Bt: n=2, and Bt+Ba: n=2, where n represents cage units). Shotgun metagenomics was then used to monitor microbiome changes every 3 days. (b) Microbial biomass (median±1 MAD) in different groups of mice across time (excluding gavaged species). (c) Microbiome diversity (Simpson) (median±1 MAD) in different groups of mice across time. Stars (‘*’) indicate timepoints where Bt+Ba group differs from other groups (one-sided Wilcoxon test p-value<0.1). (d, e, f) Reads per million (RPM) mapping to CAZymes (median±1 MAD) associated with plant cell wall/animal carbohydrate, mucin and peptidoglycan degradation, respectively, across different experimental groups and timepoints. Stars in all subfigures (‘**’) indicate timepoints where the Bt and Bt+Ba groups were significantly different from other groups (one-sided Wilcoxon test p-value<0.01).

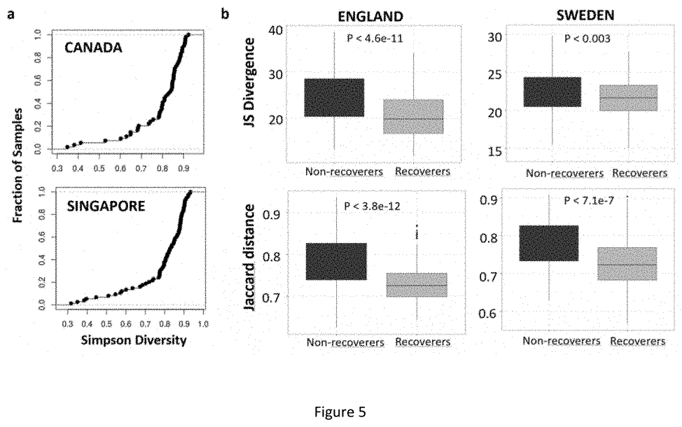

FIG. 5: Properties of microbiome recovery across cohorts. (a) Cumulative density function for Simpson diversity in the CA and SG cohorts, highlighting the large number of low diversity samples. (b) Microbiomes of recoverers are more similar to control microbiomes than for non-recoverers (two-sided Wilcoxon test; n=16 [EN], n=32 [SW] recoverers and n=24 [EN], n=23 [SW] for non-recoverers). Jensen-Shannon (J S) divergence and Jaccard distances for each sample were computed in comparison to the untreated (“control”) microbiomes in each cohort. The figures show the median values for each sample in the form of a boxplot. Boxplots are represented with center line: median; box limits: upper and lower quartiles; box whiskers represent 1.5×interquartile range or the maximum/minimum data point within the range.

FIG. 6: Enrichment of RABs during different stages of antibiotic treatment. Fold change was computed for median abundance in recovered vs non-recovered subjects per cohort and averaged across all 4 cohorts. Groups were determined manually (due to limited dimensionality) based on approximate trends and taxonomic similarity. The symbols “*”, “*” and “***” indicate p-values <0.1, <0.05 and <0.01, respectively based on two-sided Wilcoxon test comparison between recoverers (n=113) and non-recoverers (n=90).

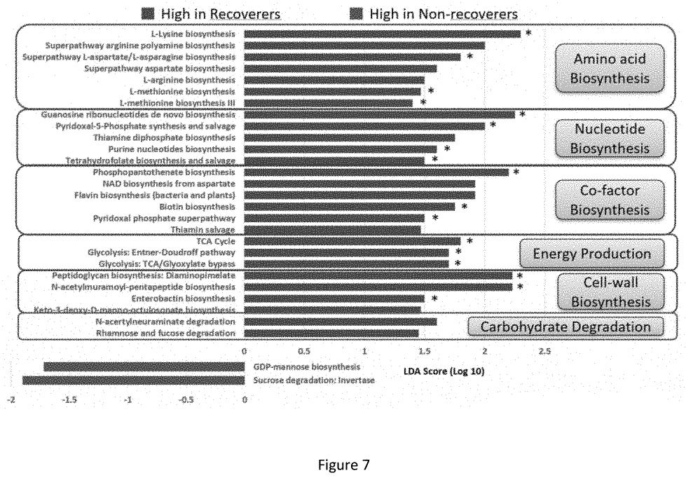

FIG. 7: Differentially abundant metagenomic functions in post-antibiotic recovery. Functional pathways enriched in the gut microbiomes of recoverers (n=17) or non-recoverers (n=12) (of the SG cohort) in the ‘Pre/Early’ and ‘During’ stages of antibiotic treatment. Note that a star (‘*’) indicates those pathways for which significant (p-values<0.05) differences were also obtained in the CA cohort. p-values were computed using the KW-rank sum test implemented within the LefSe package. Pathways were grouped into those important for energy production (in orange) and those involved in biosynthesis (in blue), highlighting the role of these two processes in microbiome recovery.

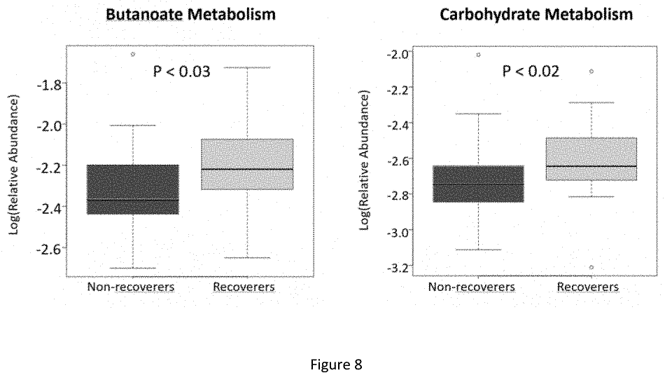

FIG. 8: Enrichment of Carbohydrate Metabolism and Butanoate Metabolism pathways in the gut microbiomes of recoverers in the EN and SW cohorts. Abundances of the various pathways in the samples belonging to the Pre/Early and During stages of treatment were inferred using PICRUSt and then compared among the recoverers (n=8 [EN], n=16 [SW]) and non-recoverers (n=11 [EN], n=12 [SW]) in these cohorts. The total-sum-scaled abundances were log-normalized and compared using two-sided Wilcoxon test. Boxplots are represented with center line: median; box limits: upper and lower quartiles; box whiskers represent 1.5×interquartile range or the maximum/minimum data point within the range.

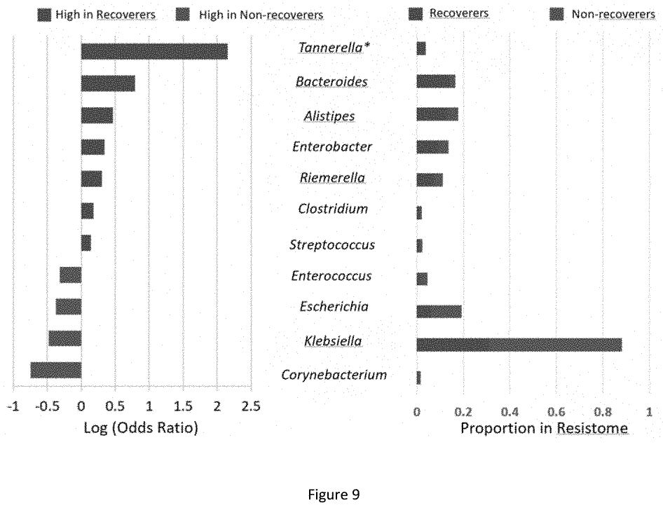

FIG. 9: Enrichment of Bacterial Genera in the Resistome. Reads belonging to the resistome were assigned to bacterial genera using Kraken (right panel) and odds ratio between groups computed to identify enriched genera (left panel; *=x² test p-value <0.05, pre- and during antibiotic timepoints). Genera with RAB species are highlighted in green. The comparisons were performed for the “Pre/Early” and “During” samples belonging to the SG and CA cohorts (n=17 [SG], 16 [CA] recoverers; n=12 [SG], 14 [CA] non-recoverers).

FIG. 10: Enrichment of Bacterial Genera in the Resistome. Reads belonging to the resistome were assigned to bacterial genera using Kraken (right panel) and odds ratio between groups computed to identify enriched genera (left panel; *=x² test p-value <0.05, pre- and during antibiotic timepoints). Genera with RAB species are highlighted in green. The comparisons were performed for the “Pre/Early” and “During” samples belonging to the SG and CA cohorts (n=17 [SG], 16 [CA] recoverers; n=12 [SG], 14 [CA] non-recoverers).

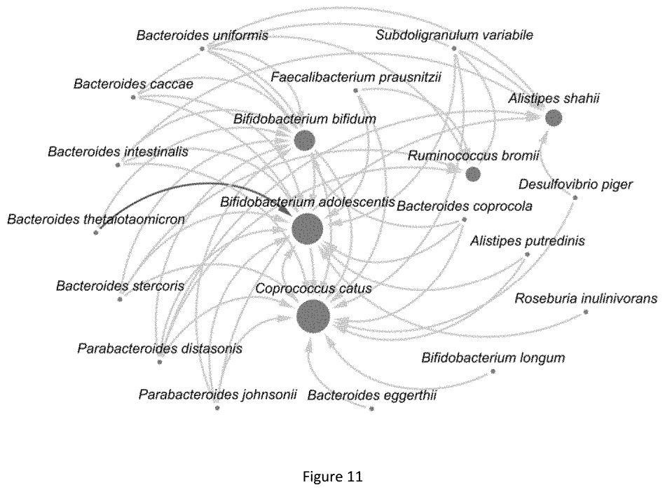

FIG. 11: Key metabolic interactions between RABs. Directed lines indicate RAB species with high metabolic support to other RAB species (top 10% of MSI values). Node sizes reflect the number of incoming edges and the red edge marks the interaction between B. thetaiotamicron and B. adolescentis which was evaluated further in an in vivo model for microbiome recovery.

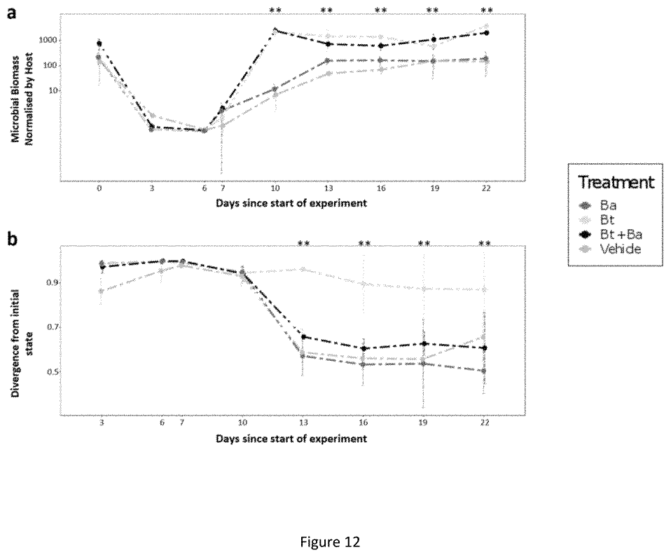

FIG. 12: Microbiome recovery profiles across treatment groups. (a) Microbial biomass (median±1 MAD) values obtained after normalizing by host reads reveal similar trajectories as plant normalized values (FIG. 4b). Stars (‘**’) indicate timepoints where the Bt and Bt+Ba groups were significantly different from other groups (one-sided Wilcoxon test p-value <0.01). (b) Median Bray-Curtis distance of species level taxonomic profiles compared to day 0 profiles, in different treatment groups and across time (median±1 MAD). Stars (‘**’) indicate timepoints where the Bt group was significantly different from other groups (one-sided Wilcoxon test p-value <0.01). For all subfigures, vehicle: n=5, Ba: n=6, Bt: n=2, and Bt+Ba: n=2, where n represents cage units.

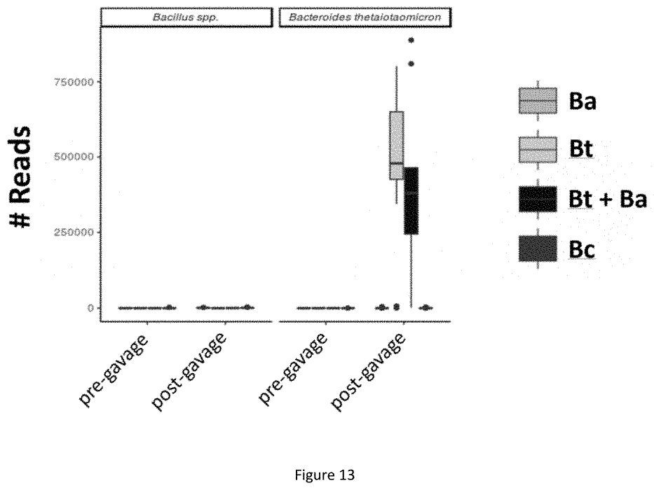

FIG. 13: Successful colonization of B. thetaiotaomicron in the mouse gut microbiome post gavage. Boxplots showing high number of B. thetaiotaomicron metagenomic reads from mouse stool after Bt gavage, but not Bacillus spp. reads after Bacillus gavage (Bc group), indicating successful colonization specific to Bt. Boxplots are represented with center line: median; box limits: upper and lower quartiles; whiskers: 1.5×interquartile range. Ba: n=18 (pre-gavage), 36 (post-gavage) samples; Bt: n=6 (pre-gavage), 12 (post-gavage) samples; Bt+Ba: n=6 (pre-gavage), 12 (post-gavage) samples; Bc: n=18 (pre-gavage), 36 (post-gavage) samples.

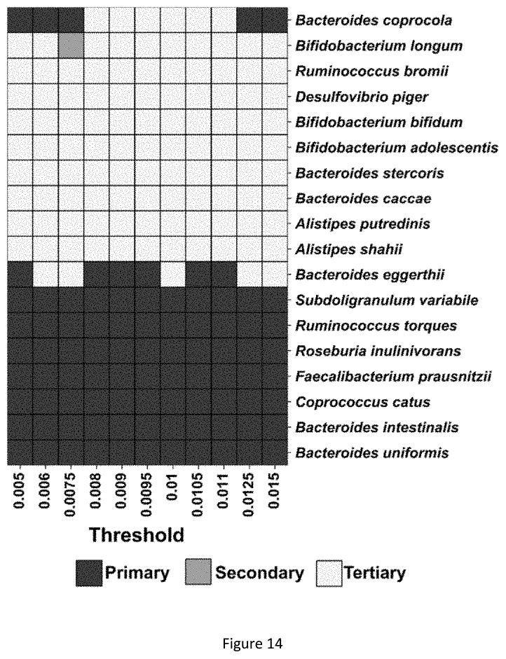

FIG. 14: Placement of RABs in the food web at different thresholds. Heatmap showing that at different thresholds (±50% from the threshold of 0.01 used for results in FIG. 3a), the position of RABs as primary, secondary and tertiary species in the food-web is retained.

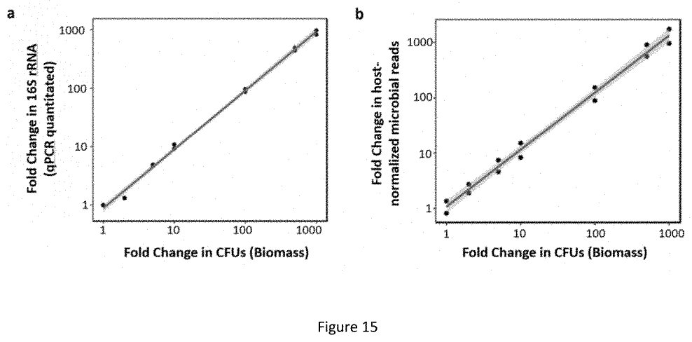

FIG. 15: Establishing validity of microbial biomass estimation using host normalized microbial read counts. (a) 16S rRNA qPCR demonstrates that the fold change in 16S rRNA copies is directly proportional to fold change in microbial biomass (CFUs), as expected. (b) Metagenomic analysis demonstrate that the fold change in host-normalized microbial reads is directly proportional to fold change in microbial biomass (CFUs). DNA from cultures of Klebsiella pneumoniae and Enterococcus faecium were mixed in equal CFU ratio, and mouse stool DNA samples were spiked in at various dilutions (1:1 to 1:1000) to achieve a wide-range of fold changes. Data shown is for two stool samples (biological replicates).

### EXAMPLE

Loss of diversity in the gut microbiome can persist for extended periods after antibiotic treatment, impacting microbiome function, antimicrobial resistance and likely host health. Despite widespread antibiotic use, our understanding of species and metabolic functions contributing to gut microbiome recovery is limited. Using data from 4 discovery cohorts in 3 continents comprising >500 microbiome profiles from 117 subjects, 21 bacterial species exhibiting robust association with ecological recovery post antibiotic therapy were identified. Functional and growth-rate analysis showed that recovery is supported by enrichment in specific carbohydrate degradation and energy production pathways. Association rule mining on 782 microbiome profiles from the MEDUSA database enabled reconstruction of the gut microbial ‘food-web’, identifying many recovery-associated bacteria (RABs) as keystone species, with ability to use host and diet-derived energy sources, and support repopulation of other gut species. Experiments in a mouse model recapitulated the ability of RABs (Bacteroides thetaiotamicron and Bifidobacterium adolescentis) to promote recovery with synergistic effects, providing a two orders of magnitude boost to microbial abundance in early time-points and faster maturation of microbial diversity. The identification of specific species and metabolic functions promoting recovery opens up opportunities for rationally determining pre-/probiotic formulations offering protection from long-term consequences of frequent antibiotic usage.

METHODS

Study Populations

(a) Singapore: The Singaporean cohort (‘SG’; manuscript in preparation) is a natural history cohort consisting of individuals admitted to Tan Tock Seng Hospital (TTSH) in Singapore and prescribed antibiotics for 1-2 weeks (primarily Co-amoxiclav and Clarithromycin; Table 1). Stool samples were collected as soon as possible after admission (pre-/early: <3 days into treatment), during and up to 3 months after antibiotic usage. The study was approved by the Institutional Review Board at TTSH (DSRB 2013/00769).

(b) Canada: Shotgun metagenomic datasets for a Canadian cohort (‘CA’) were obtained from the European Nucleotide Archive database (Study Accession Number: PRJEB8094; Table 1). The study analyzed fecal samples from healthy individuals who were administered antibiotics (Cefprozil; three timepoints: pre-antibiotic day 0, during treatment day 7 and post treatment day 90).

(c) England and Sweden: 16S rRNA sequencing datasets for an English and a Swedish cohort (‘EN’, ‘SW’) were obtained from the NCBI short read archive (Project ID: SRP057504; Table 1). In both cohorts, healthy volunteers were given antibiotics (EN: Amoxicillin, SW: Clindamycin/Ciprofloxacin) and fecal samples analyzed for day 0 (pre-antibiotic), day 7 (during treatment) and for one and two month follow-ups (post treatment).

(d) NUH A prospective cohort of young Chinese adults was recruited to study the impact of antibiotics on the gut microbiome at the National University Hospital, located in Singapore, (NUH; 5-day course of Co-amoxiclav; manuscript in preparation). Stool samples were collected before (day 0), during (day 1-5) and after antibiotic cessation (day 8 and day 28). The study was approved by the Institutional Review Board at NUH (DSRB 2012/00776).

For the CA, EN and SW cohorts, all antibiotic treated subjects with data from the 3 treatment stages were further analyzed to identify recovery associated bacterial taxa and functions.

TABLE 1

| | No. of | | | |
|---|---|---|---|---|
| | Subjects/ | | | |
| Cohort | Samples | Sequencing | Age Range | Antibiotics Used |
| Singapore | 27/129 | Shotgun | 32-81 | Primarily Co- |
| (SG) | | Metagenomic | | amoxiclav and |
| | | | | Clarithromycin |
| Canada | 24/72 | Shotgun | 21-35 | Cefprozil |
| (CA) | | Metagenomic | | |
| England | 37/219 | 16S rRNA | 24-26 | Amoxicillin |
| (EN) | | | | |
| Sweden | 29/173 | 16S rRNA | 22-30 | Clindamycin/ |
| (SW) | | | | Ciprofloxacin |
| NUH | 24/72 | Shotgun | 23-40 | Co-amoxiclav |
| | | Metagenomic | | |

DNA Extraction and Sequencing for SG and NUH Cohorts

Extraction of DNA from stool samples was carried out using PowerSoil DNA Isolation Kit (MoBio Laboratories, California, USA) with minor modifications to the manufacturer's protocol (volume of solutions C2, C3 and C4 were doubled and centrifugation time was extended to twice the original duration). Purified DNA was eluted in 80 μl of Solution C6. DNA libraries were prepared by using 20 ng of extracted DNA re-suspended in a volume of 50 μl and subjected to shearing using Adaptive Focused Acoustics™ (Covaris, Mass., USA) with the following parameters; Duty Factor: 30%, Peak Incident Power (PIP): 450, 200 cycles per burst, Treatment Time: 240s. Sheared DNA was cleaned up with 1.5×Agencourt AMPure XP beads (A63882, Beckman Coulter, Calif., USA). End-repair, A-addition and adapter ligation was carried out using the Gene Read DNA Library I Core Kit (Qiagen, Hilden, Germany) according to the manufacturer's protocol. Custom barcode adapters (see Table 2 below) were used in place of GeneRead Adapter I Set for adapter ligation. DNA libraries were cleaned up twice using 1.5×Agencourt AMPure XP beads (A63882, Beckman Coulter, Calif., USA) before enrichment of libraries using the protocol adapted from Multiplexing Sample Preparation Oligonucleotide kit (Illumina, Calif., USA). Enrichment PCR was carried out with PE 1.0 and custom index-primers (Table 3) for 14 cycles. Libraries were quantified using Agilent Bioanalyzer and prepared with Agilent DNA1000 Kit (Agilent Technologies, California, USA), pooled in equimolar concentrations. Sequencing of the samples was performed using the Illumina HiSeq 2500 (Illumina, Calif., USA) sequencing instrument to generate >80 million 2×101 bp reads on average.

TABLE 3

| | 1^{st} strand: 5′P-GATCGGAAGA |
|---|---|
| | GCACACGTCT (SEQ ID NO 1) |
| | 2^{nd} strand: 5′ACACTCTTTCCC |
| Barcode adapter, | TACACGACGCTCTTCCGATCT |
| double stranded | (SEQ ID NO 2) |
| PE 1.0 | 5′AATGATACGGCGACCACCGAGATCTACA |
| | CTCTTTCCCTACACGACGCTCTTCCGATC* |
| | T (SEQ ID NO 3) |
| Index Primer | 5′CAAGCAGAAGACGGCATACGAGATXXXX |
| | XXXXGTGACTGGAGTTCAGACGTGTGCTCT |
| | TCCGATC*T (SEQ ID NO 4) |
| 16S Forward | 5′ACTCCTACGGGAGGCAGC |
| | (SEQ ID NO 5) |
| 16S Reverse | 5′TTACCGCGGCTGCTGGCAC |
| | (SEQ ID NO 6) |
| gBLOCK: | 5′GGCCCAGACTCCTACGGGAGGCAGCAGT |
| | AGGGAATCTTCGGCAATGGACGGAAGTCTG |
| | ACCGAGCAACGCCGCGTGAGTGAAGAAGGT |
| | TTTCGGATCGTAAAGCTCTGTTGTAAGAGA |
| | AGAACGAGTGTGAGAGTGGAAAGTTCACAC |
| | TGTGACGGTATCTTACCAGAAAGGGACGGC |
| | TAACTACGTGCCAGCAGCCGCGGTAATACG |
| | TAGGTCCCGAG (SEQ ID NO 7) |

Taxonomic and Functional Profiling for all Cohorts

For metagenomic sequencing datasets (CA, SG and NUH cohorts) raw reads were quality filtered and trimmed using default options in famas (https://github.com/andreas-wilm/famas). Reads that are potentially from human DNA were removed by mapping to the hg19 reference using BWA-MEM (default parameters; coverage >80% of read). The remaining reads were used for taxonomic profiling using MetaPhlAn with default parameters (Supplementary Data 1). Functional profiles for the metagenomes were obtained using the HUMAnN2 program (Supplementary Data 3).

The Supplementary Data referenced in this application may be assessed at https://www.nature.com/articles/s41559-020-1236-0 #additional-information.

For the 16S rRNA sequencing datasets (EN and SW cohorts) taxonomic classification was done by mapping reads to the SILVA database (v123) using blastn. For each read, the species corresponding to the best hit (with identity >97% and query coverage >95%) was obtained and was taken as the source species of the read. In the case of multiple hits, the source taxon was computed as the Lowest Common Ancestor of the hit species. Reads assigned to each taxon were aggregated to obtain a relative abundance profile for each sample (Supplementary Data 1). PICRUSt was used to infer KEGG pathway abundances from the corresponding taxonomic profiles (Supplementary Data 3).

Identification of Recovery Associated Bacterial Taxa and Functions

Individuals were classified as ‘recoverers’ and ‘non-recoverers’ in each cohort to enable cohort-specific association analysis and identification of recovery associated bacterial taxa and functions. As post-antibiotic microbiomes may not necessarily resemble the pre-antibiotic state for an individual (e.g. due to enterotype switching), the post-treatment gut microbial diversity (species-level; Simpson) was used to define recoverers and stratify subjects into balanced groups (median threshold). Samples within a 10% window of the interquartile range from the median were marked as having indeterminate status and excluded from further analysis. A two-stage approach was used to combine results from all cohorts to sensitively identify recovery associated taxa and a cross-cohort validation strategy was used to identify taxa that are significant in at least 2 out of 4 cohorts. In stage 1, a non-parametric test was used within each cohort to identify candidate taxa (one-sided Wilcoxon test). The resulting p-values were merged across cohorts to compute a combined p-value using Fisher's method and filtered with a FDR adjusted p-value threshold of 0.01 (Benjamini-Hochberg method). Next, in stage 2, cohort-specific FDR adjusted p-values (Benjamini-Hochberg method) were re-computed for this subset of taxa and only taxa with consistent (in terms of direction of change) significant associations (FDR<0.05) in at least 2 cohorts were retained. This analysis was done within each treatment stage (pre-, during and post-antibiotics) as well as jointly to increase sensitivity in identifying recovery associated taxa regardless of treatment stage.

Functional profiles computed with HUMAnN2 were compared between recoverers and non-recoverers in the SG and CA cohorts using the linear discriminant analysis approach in LEfSe (version 1.1.0) to identify differentially abundant pathways.

Microbial Community Growth Rate Analysis

An in silico approach, originally proposed by Korem et al, was used to compute the skew of DNA copy number starting from around the origin of replication to the termination region (peak-to-trough ration or PTR), as an estimate of growth rates for individual species in the microbiome from shotgun metagenomic data (PTRC1.1: https://genie.weizmann.ac.il/software/bac_growth.html, default parameters). The community growth rate (CGR) for each sample was then computed from the common species in the community (PTR values in >50% of samples) as the median PTR value (PTR set to lower-bound of 1 when not available; Suppl. Data File 5).

Profiling of Carbohydrate Active Enzymes (CAZymes)

An in-house nucleotide gene database for CAZymes was created by downloading sequences from NCBI corresponding to Accession IDs for different CAZyme families annotated in dbCAN (http.//csbl.bmb.uga.edu/dbCAN/). Metagenomic reads were mapped to this database for each sample with BWA-MEM (default parameters) to compute the fraction of reads mapping to the CAZyme gene per kbp per million reads in the metagenome (RPKM). Results were aggregated for each CAZyme family based on values for individual CAZyme genes belonging to a family.

Analysis of Antibiotic Resistance Genes within Gut Microbiomes

Resistome profiling within a microbiome was performed similarly by mapping metagenomic reads using BWA-MEM (default parameters) to the ARG-ANNOT database, and calculating the fraction of reads mapping to a resistance gene per kbp per million reads of the metagenome (RPKM). Kraken was used with default parameters to obtain the taxonomic classification of reads and thus obtain the relative representation of different taxonomic groups within the resistome.

Clustering of Species Based on their Carbohydrate Degradation Profiles

The substrate-specificities of different Glycoside hydrolase (GH) and Polysaccharide lyase (PL) families were obtained from previous studies. These included substrates such as plant cell wall carbohydrates, animal carbohydrates, peptidoglycans, fungal carbohydrates, sucrose/fructose, dextran, starch/glycogen and mucins. Copy number annotations for each Gil and PL family in 137 bacterial species were obtained from a previous genome scale analysis of CAZymes in species belonging to the human gut microbiome. Copy numbers of GH/PL genes within each of the 8 substrate specificities were aggregated and normalized to obtain an overall carbohydrate degradation profile for each bacterial species. Degradation profiles were then clustered using hierarchical clustering (‘hclust’ function in R with Euclidean distance and complete linkage clustering) to group species based on their enzyme repertoire for different categories of carbohydrates. Association of the identified recovery associated bacteria to one or more of these clusters was then evaluated using Fisher's exact test.

Construction of Microbial Food-Web Using Association Rule Mining

To identify directed associations between bacterial species where the presence of one is important for the presence of another (but not vice versa), a data-mining technique called ‘association rule mining’ was applied to a large public collection of gut microbiome profiles in the MEDUSA database (782 gut microbiome profiles from USA, China and Europe). To convert relative abundance profiles from MEDUSA into presence-absence profiles (1 if a species is present and 0 otherwise),

$$relative abundances < {min}_{j} {a}_{ij} + 0.01 ⨯ ( Q 95 {a}_{ij} - {min}_{j} {a}_{ij} ) ,$$

i.e. within 1% of the minimum relative abundance values aᵢⱼ for species i across subjects j (Q95 or 95% percentile was used instead of max to improve robustness to outliers), were assumed to be due to technical noise. Note that overall results were confirmed to be robust (in terms RAB placement) to a range of threshold values (±50% of original values; FIG. 14). Binary association rules between species were then inferred using the apriori algorithm implemented in the Python package ‘efficient_apriore’ (using Confidence threshold of 0.95 and Support threshold of 0.05). After removal of transitive edges and symmetric relationships, a total of 1166 directed association edges remained across 266 species (Supplementary Data 6). Association edges and corresponding nodes for species were plotted using the hierarchical layout of Cytoscape, where the hierarchical level of a species is influenced by the difference between the number of outgoing and incoming edges.

Metabolic Interaction Analysis

Genome-scale metabolic models (GSMMs) for RABs and control species were downloaded from the AGORA database (v1.03). Metabolic interactions were quantified by computing the Metabolic Support Index (MSI) which quantifies the percentage of metabolic reactions in an organism that become feasible in the presence of another organism. All simulations were conducted under anoxic conditions with high-fiber diet, and mucin and bile acid derived metabolite supplementation. Species pairs with high MSI values (top 10%) were visualized using Cytoscape (v3.7.2).

Promoting Microbiome Recovery in a Mouse Model

Ethics statement: Mouse experimental protocols were reviewed, approved and carried out in strict accordance to the recommendations by the Institutional Animal Care and Use Committee (IACUC) in the animal facility at Comparative Medicine, National University of Singapore (NUS). The care and use of animals for research and teaching in NUS is bound by the Singapore Animals and Birds Act, Animals and Birds (Care and Use of Animals for Scientific Purposes) Rules 2004, and is carried out in accordance with the National Advisory Committee for Laboratory Animal Research (NACLAR) Guidelines. NUS is an AAALAC-accredited institution. For this study, animals were used under Protocol R15-0135 as approved by the NUS IACUC.

Bacterial strains and culture conditions: Lyophilized probiotic strains (ATCC 29148 Bacteroides thetaiotaomicron, DSM 20083 Bifidobacterium adolescentis) were revived in TSB media supplemented with 5% defibrinated sheep blood under anaerobic conditions at 37° C. Upon revival, B. thetaiotaomicron was subcultured and maintained in TYG media, whereas B. adolescentis and an environmental Bacillus isolate were subcultured and maintained in BHI media.

Antibiotic administration and inoculation with test strains: Eight-week-old C57BL/6J male mice from a single breeding colony were purchased from InVivos Singapore. The mice were gavaged individually with 2.5 mg ampicillin sodium salt (Sigma Aldrich) prepared in 1×PBS per day for 5 days using flexible sterile plastic feeding tubes (Instech Labs) under specific pathogen-free conditions. Upon cessation of antibiotic treatment, mice were allowed to recover for 24 hours, before the cages of mice (two mice per cage; two experimental batches) were each orally inoculated with: A) 5×10⁷ CFUs B. thetaiotaomicron, B) 5×10⁷ CFUs Bacillus spp., C) 5×10⁷ CFUs B. adolescentis, D) 5×10⁷ CFUs B. thetaiotaomicron+5×10⁷ CFUs B. adolescentis, E) 5×10⁷ CFUs Bacillus spp.+5×10⁷ CFUs B. adolescentis, or F) phosphate-buffered saline (PBS). Mice were kept on a 12 h light/dark cycle, and water and autoclaved standard chow diet were provided ad libitum. Mice were caged in pairs in transparent plastic cages with corn cob bedding that had been pre-sterilised by autoclaving. Only mice in Bt/Bt+Ba cages where gavage was successful to result in detection in fecal samples were used for further analyses. Strains were transported from anaerobic chamber to animal facility via anaerobic “balch-type” culture tubes with aluminum seals (Chemglass Life Sciences, New Jersey, USA).

Fecal sample collection and DNA extraction: Fecal pellets were freshly collected as a cage unit (two mice per cage) over multiple times points: before antibiotic treatment (Day 0), mid-point of antibiotic treatment (Day 3), end-point of antibiotic treatment (Day 6), 1-day post-gavage (Day 7), 4-days post-gavage (Day 10), 7-days post-gavage (Day 13), 10-days post-gavage (Day 16), 13-days post-gavage (Day 19) and 16-days post-gavage (Day 22). Total bacterial DNA was extracted from fecal samples using the PowerSoil DNA isolation kit (MoBio Laboratories) according to the manufacturer's instructions.

Library preparation and deep sequencing: DNA libraries were prepared and sequenced with the same kits and workflow as used for the SG and NUH cohorts, except that the input DNA amount was 50 ng.

Taxonomic profiling: For obtaining the taxonomic profiles of the mouse gut metagenomes, reads were mapped to the NR database using DIAMOND. The taxonomic classification of each sequence was then obtained by using the LCA-based approach in MEGAN (default parameters, minimum score of 50).

Calculation of microbial biomass: Bacterial biomass (up to a constant factor) was estimated by taking all reads classified to bacterial taxa and normalizing by non-microbial reads. Specifically, plant or host-derived reads were used, respectively, based on the assumption that the absolute amounts of their DNA would remain roughly constant in the analyzed mouse fecal samples. Similar trends were observed for both forms of normalization (default=plant normalized), normalization based abundances were found to correlate with qPCR estimates (plant normalized, r=0.73, p-value=10⁴; host normalized, r=0.82, p-value=3.5×10⁻⁶), and the observed differences between Bt and Bt+Ba groups versus other groups were also validated using qPCR (day 10, fold-change=94-170×). Note that sequencing based biomass estimates have the advantage that they allow us to subtract reads belonging to the gavaged species and are also not affected due to variations in 16S rRNA copy number across taxa. This approach was also further validated based on spike-in of isolate DNA into mouse stool samples showing that (i) qPCR based measurement of 16S rRNA DNA copies correlates highly with microbial CFUs (slope=0.98, R²=1.0; FIG. 15a), (ii) Metagenomic sequencing based calculation of host-normalized microbial reads accurately quantitated varying microbial CFUs (FIG. 15b).

qPCR Analysis: Absolute quantification of the 16S rRNA gene was done by quantitative PCR (qPCR). A pair of universal 16S bacterial primers were used to amplify DNA extracted from the six different treatment groups on days 0, 3, 10 and 13 (Table 2). Reactions were prepared on a 384-well plate, in triplicates, using 5 μL of PowerUp SYBR Green Master Mix (Thermo Fisher Scientific, Massachusetts, USA), 0.5 μL of 5 μM primers and 1 μL of 10×diluted DNA, in a total volume of 10 μL for each reaction. The ViiA 7 Real-Time PCR System (Thermo Fisher Scientific, Massachusetts, USA) was used for qPCR with the following amplification parameters: 1 cycle of 95° C. for 2 min, 40 cycles of 95° C. for 15 s, 60° C. for 15 s, and 72° C. for 1 min. A standard curve was created using serial dilution of synthesized double-stranded DNA oligomers (gBLOCK, Integrated DNA Technologies, Inc., Iowa, USA; Table 2) to convert CT values to copy numbers. Copy numbers from day 0 were used to scale bacterial abundances to the same starting baseline.

Data Availability

Illumina sequencing data for this study (mouse models) is available from the Sequence Read Archive under project ID SRP142225. Samples are labelled in SRA with a shorthand, e.g. PBS6D22, where “PBS” represents gavage condition, “6” represents cage number and “D22” represents day of sampling.

Code Availability

Analysis scripts used for generating the figures in this study are available at https://github.com/CSB5/Recovery_Determinants_Study.

RESULTS

Robust Identification of Microbial Taxa Associated with Gut Microbiome Recovery

In order to identify microbial markers associated with gut microbiome recovery, longitudinal data from 4 cohorts (a total of 117 individuals with >500 samples; Methods) were assembled and systematically analyzed. These cohorts represent individuals from 4 countries on 3 continents (Singapore, Canada, England, Sweden), a range of age groups (21-81) and using different classes of antibiotics, allowing us to infer common factors associated with microbiome recovery (Table 1). Data from the Singaporean cohort was newly generated and analyzed (deep shotgun metagenomic sequencing of 74 samples; >80 million reads on average), involving mostly elderly subjects receiving inpatient antibiotic treatment (Supplementary Data 1). Each cohort was analyzed independently to account for cohort-specific biases, and the results were aggregated using a cross-cohort validation approach to only identify microbial taxa that were independently associated with recovery in at least 2 cohorts (Methods).

To stratify individuals based on their recovery status, it is noted that many individuals exhibited a U-shaped profile for gut microbial diversity, with a significant drop in diversity during antibiotic treatment, but with recovery of diversity in post-treatment timepoints (‘recoverers’, FIG. 1a). A subset of individuals, however, continued to have low gut microbial diversity even 3 months post antibiotics (‘non-recoverers’, FIG. 1a), contrasting with those at the other end of the diversity spectrum (FIG. 5a). Therefore subjects have been stratified based on post-antibiotic microbial diversity as a readily defined reference-free metric for recovery across cohorts (Methods). This metric correlated well with alternative definitions, for e.g. as expected, post-antibiotic microbiomes for recoverers were much more similar to healthy control microbiomes overall, compared to non-recoverers (one-sided Wilcoxon p-value<0.001; FIG. 1b, c). This pattern was seen to be consistent across cohorts and using different diversity metrics (FIG. 5b). Recoverers and non-recoverers also did not have significant differences in microbial diversity in the pre-antibiotic state (Wilcoxon p-value>0.05).

To determine microbial taxa with a role in microbiome recovery, a two-stage approach and cross-cohort validation strategy was used to increase sensitivity and specificity of the association analysis across all timepoints (Methods; Supplementary Data 2; 34 bacterial species in stage 1). In total, 21 microbial species were identified to be significantly associated with microbiome recovery in at least 2 cohorts (Recovery Associated Bacteria-RAB; Table 2), with 10 species identified in 3 cohorts and 1 in all 4 cohorts (Bacteroides uniformis; FIG. 1d, using data for all timepoints). Variability across cohorts may reflect differences in diet, environment and antibiotics used, while genus-level consistencies (e.g. Bacteroides species; FIG. 1d; Table 2) may reflect functional redundancies in associated species. While some RABs are common gut bacteria (e.g. Alistipes putredinis), are known to have host-beneficial functions (e.g. Faecalibacterium prausnitzii) and have been observed to be depleted in disease states (e.g. B. uniformis), others are more variably distributed, with limited understanding of their function in the gut microbiome, and their role in gut microbiome recovery after antibiotic treatment being unknown (Table 2). The distribution of most RABs across recoverers and non-recoverers suggests that their abundance, rather than their presence or absence, likely contributes to the recovery process. In addition, as no RAB segregates recoverers and non-recoverers on its own in any cohort, the combined influence of multiple RABs likely determines successful microbiome recovery.

TABLE 2

| | | | | | Known functions | |
|---|---|---|---|---|---|---|
| | Cohort-specific FDR adjusted p-value | | | | or associations in | NUH p- |
| Species | Canada | England | Sweden | S’pore | gut microbiome | value |
| Bacteroides | 0.009 | 0.003 | 0.005 | 0.019 | Negatively | 0.354 |
| uniformis | | | | | associated with | |
| | | | | | obesity | |
| Alistipes | 0.002 | 0.737 | 0.011 | <0.001 | Associated with | 0.011 |
| putredinis | | | | | weight loss in | |
| | | | | | obese individuals | |
| Alistipes shahii | 0.009 | 0.018 | 0.113 | <0.001 | | 0.026 |
| Bacteroides | 0.002 | 0.953 | 0.011 | 0.002 | Diverse | 0.007 |
| thetaiotaomicron | | | | | carbohydrate | |
| | | | | | degrading | |
| | | | | | enzymes | |
| Parabacteroides | 0.004 | 0.927 | 0.005 | <0.001 | Carbohydrate | 0.218 |
| distasonis | | | | | degrading | |
| Coprococcus | 0.034 | 0.003 | 0.022 | 0.492 | | 0.096 |
| catus | | | | | | |
| Bifidobacterium | 0.003 | 0.014 | 0.342 | 0.006 | Known probiotic | 0.008 |
| adolescentis | | | | | | |
| Ruminococcus | 0.023 | 0.014 | 0.477 | 0.046 | | 0.138 |
| bromii | | | | | | |
| Subdoligranulum | 0.002 | 0.039 | 0.039 | 0.401 | Produces butyrate | 0.197 |
| variabile | | | | | | |
| Bacteroides | 0.351 | 0.013 | 0.011 | 0.050 | | 0.977 |
| stercoris | | | | | | |
| Bacteroides | 0.087 | 0.570 | 0.016 | 0.022 | | 0.039 |
| eggerthii | | | | | | |
| Bacteroides | 0.075 | 0.003 | 0.933 | 0.015 | | 0.030 |
| coprocola | | | | | | |
| Bifidobacterium | 0.049 | 0.737 | 0.239 | 0.013 | | 0.327 |
| bifidum | | | | | | |
| Roseburia | 0.133 | 0.024 | 0.022 | 0.775 | Produces butyrate | 0.308 |
| inulinivorans | | | | | | |
| Bacteroides | 0.001 | 0.737 | 0.156 | <0.001 | Negatively | 0.003 |
| caccae | | | | | associated with | |
| | | | | | obesity | |
| Faecalibacterium | 0.001 | 0.013 | 0.150 | 0.504 | Butyrate | 0.081 |
| prausnitzii | | | | | producing with | |
| | | | | | anti-inflammatory | |
| | | | | | properties | |
| Ruminococcus | 0.775 | 0.013 | 0.662 | 0.015 | Degrades mucin | 0.003 |
| torques | | | | | | |
| Bifidobacterium | 0.033 | 0.737 | 0.150 | 0.021 | Known probiotic | 0.378 |
| longum | | | | | | |
| Bacteroides | 0.002 | 0.737 | 0.574 | <0.001 | Carbohydrate | 0.377 |
| intestinalis | | | | | degrading; | |
| | | | | | Negatively | |
| | | | | | associated with | |
| | | | | | obesity | |
| Desulfovibrio | 0.223 | 0.149 | 0.011 | 0.023 | Sulfate-reducing | 0.055 |
| piger | | | | | bacteria | |
| Parabacteroides | 0.005 | 0.439 | 0.933 | 0.012 | | 0.030 |
| johnsonii | | | | | | |

RABs were initially identified across treatment stages (pre-, during and post-antibiotics; Methods) to capture species that may contribute to recovery at any stage. Abundance patterns of RABs were then investigated across stages and it was noted that while some were 2-4× more abundant in recoverers before treatment (e.g. B. uniformis), others were enriched in later timepoints, indicating that they may play a secondary or synergistic role in recovery (FIG. 6; e.g. F. prausnitzii), and that combinatorial effects across treatment stages may play a role in recovery. Interestingly, no RABs were depleted in the gut microbiomes of recoverers versus non-recoverers, indicating that they do not have specific inhibitory roles. Training of machine learning models across cohorts showed that post-antibiotic recovery status can be predicted to an extent using pre-antibiotic taxonomic abundances for an individual (70.4% accuracy). Machine learning models to predict recovery status may be used. For example, to test the ability to infer recovery status using microbial abundances before antibiotic treatment (with and without cohort labels; only microbes with mean relative abundance >0.5% were used), attempts were made to build a classifier with various machine learning models, including random forest (R package “randomForest”), linear discriminant analysis (R package “MASS”), sparse logistic regression (R package “glmnet”), and conditional inference tree (R package “ctree”). The models were evaluated with default parameters using leave-one-out cross validation (R package “caret”) and the accuracy for the best model (conditional inference tree) was reported.

A fifth cohort of healthy young adults in Singapore taking antibiotics (NUH, Table 1) was enlisted, whose metagenomes were not sequenced at the point of initial association analysis with the original four cohorts, to study the consistency of RABs across cohorts. Overall, 12 out of 21 RAB species were significantly associated (one-sided Wilcoxon p-value <0.1) in the new cohort as well, similar to the overlap of the four original cohorts with RAB species (10-17 species, Table 2), confirming the robustness of associations despite differences in age, location and antibiotics used. In addition, incorporation of the fifth cohort in the cross-cohort association analysis only increase the list of RABs by 2, highlighting the consistency and reproducibility of this list.

Enrichment in Carbohydrate Degradation and Energy Metabolism Pathways Links RABs with Microbial Community Growth and Recovery

To study microbial functions that link RABs to microbiome recovery, all differentially abundant gene families and pathways in the pre- and during treatment metagenomes of recoverers and non-recoverers (CA and SG cohorts, Methods; FDR adjusted p-value<0.1 and LDA score >1.25; Supplementary Data 3) were systematically identified. This analysis highlighted a core set of growth-associated pathways pertaining to the biosynthesis of amino acids, nucleotides, co-factors and cell wall constituents (FIG. 7). In addition, pathways involved in carbohydrate degradation and energy production were also significantly over-represented in the gut microbiomes of recoverers. Analysis of inferred pathway abundances from 16S rRNA profiles in the pre- and during treatment stages of the English and Swedish cohorts further confirmed these associations (carbohydrate and butanoate metabolism, Wilcoxon test p-value<0.05; FIG. 8; Supplementary Data 3). In comparison, analysis of resistomes of recoverers and non-recoverers in the pre- and during treatment stages did not show any significant enrichment for RAB species indicating that antibiotic resistance functions do not, in general, explain the taxonomic differences observed (see Methods below; FIG. 9).

To further understand the role of carbohydrate processing functions in microbiome recovery, carbohydrate-active enzyme families were annotated in RABs and the gut metagenomes of recoverers and non-recoverers (based on CAZyme families, Methods). Overall, RABs exhibited a significant enrichment for CAZyme families compared to non-RABs (two-sided Wilcoxon test p-value<0.001; FIG. 2a), though this does not seem to be a necessary or sufficient condition for identification as a RAB. The enrichment of CAZyme families in RABs was also reflected at the community level where the metagenomes of recoverers at all timepoints were enriched in CAZyme families compared to non-recoverers (one-sided Wilcoxon test p-value<0.001 and <0.05 for CA and SG respectively; FIG. 2b; Supplementary Data 4), consistent with enriched pathways in FIG. 7.

Linking the two major classes of pathways enriched in recoverers versus non-recoverers, it was hypothesized that in broad terms, higher carbohydrate metabolism capabilities in RABs could enable better nutritional harvest, thus enhancing biosynthesis and microbial growth (FIG. 7), and subsequent recovery of gut microbial diversity and biomass (FIG. 2). Using in silico estimates of community growth rates (from DNA coverage skews in replicating cells) from metagenomic data (Supplementary Data 5), it was observed that recoverers exhibited higher microbial community growth rate overall than non-recoverers across all stages of antibiotic treatment (one-sided Wilcoxon test p-value<0.001 and <0.05 for CA and SG cohorts, respectively; FIG. 2c). Additionally, it was noted that the pre- and during treatment abundance of RABs had a significantly higher correlation with post-treatment community growth rate across individuals (one-sided Wilcoxon test p-value<0.001 for CA cohort; FIG. 2d). Finally, in both the CA and SG cohorts, community growth rate at all timepoints was positively correlated with the number of CAZyme families (for CA, r=0.729; for SG, r=0.556; p-value<0.001; FIG. 2e). Taken together, these analyses consistently link together enrichment in RABs, carbohydrate degradation potential, microbial community growth rate and microbiome recovery as successive steps in a plausible mechanism for how RABs promote recovery.

Specific Carbohydrate Degradation Functions Define the Role of RABs in the Gut Microbial Food-Web

Carbohydrate active enzymes can be varied in their function and their differential and combinatorial usage by RABs could contribute to microbiome recovery. To study this, a set of 137 bacterial genomes annotated for their CAZyme repertoire was clustered based on their genome-wide profiles of substrate-specific enzyme copy numbers to obtain 5 distinct clusters (FIG. 10). Interestingly, RABs were primarily observed to aggregate in 2 out of the 5 clusters, with significant enrichment in cluster 1 containing genomes abundant in host (mucins) as well as diet-derived (plant and animal) carbohydrate degrading enzymes (Fisher's exact test p-value<0.001). The ability to degrade mucins is key for bacterial colonization of the intestine, and may assist some RABs in seeding the recovery process. While a few RABs fall in cluster 2 that is characterized by diet-derived (plant and animal) carbohydrate degrading enzymes, clusters 3, 4 and 5 (Starch, Fungal carbohydrate and Peptidoglycan degradation, respectively) were sparsely represented, highlighting the importance of specific carbohydrate degradation processes in microbiome recovery.

The recovery of many natural ecosystems is driven by ecological interactions and it was hypothesized that a similar ‘food-web’ of cross-feeding between RABs and other constituents of the gut microbiome is important for microbiome recovery. As experimental information about the gut microbial food-web is sparse, a data-driven approach was developed based on association rule mining (782 microbiome profiles from the MEDUSA database; Methods) to identify dependency relationships between bacteria in the gut microbiome (A→B), where the presence of species B appears conditional on the presence of species A (but not vice versa). The resulting network contains 1,166 directed edges linking 266 bacterial species, identified directly from gut microbiome data (Supplementary Data 6), and recapitulating several known cross-feeding interactions. (e.g. Bacteroides species and group C. coccoides species).

It has been noted in the bacterial food-web that a few species mostly have outgoing edges, indicating that they are essential for the presence of other species, while many species have mostly incoming edges highlighting their dependence on the presence of many other species. Based on this, the network was visualized by sorting species based on the difference in outgoing to incoming edges (bottom to top), revealing a pyramidal web structure (with RAB nodes highlighted, FIG. 3a). Interestingly, many RABs belonging to cluster 1, and correspondingly enriched in mucin degrading enzymes, were clustered in the bottom third of this network (denoted as primary species). No RABs were found in the middle third of the network (secondary species), while RABs in the top third of the network belong to a diverse set of CAZyme clusters (tertiary species). These observations are in agreement with the ecological expectation that while some RABs should be keystone species that are essential to triggering the repopulation effect (primary species), others play a synergistic role in later stages or serve as indicator species for ecological recovery (tertiary species).

Overall, the carbohydrate degradation profiles of RABs and their organization in the food-web is consistent with a model (FIG. 3b) where: (i) primary RABs employ their mucin degrading capabilities to successfully colonize/recolonize the gut epithelium; some of the primary RABs also serve as specialists in breaking down complex diet-derived carbohydrates (e.g. B. uniformis), (ii) this helps initiate a chain of cross-feeding interactions that support the repopulation of other bacteria (secondary or tertiary species) that cannot degrade mucins and/or are dependent on the breakdown of complex carbohydrates into simple sugars, (iii) as the microbial community repopulates, some RABs (e.g. F. prausnitizii and Roseburia species) contribute to production of SCFAs that in turn provide energy for colonocytes, and (iv) the resulting increased production of mucin creates a positive feedback loop that drives faster recovery of microbial biomass. The overall effect is the rebuilding of a food-web in the gut microbial ecosystem to support a diverse community concurrently and is distinct from the microbial succession processes that have been described in other contexts.

A Mouse Model of Microbiome Recovery Recapitulates Synergy Between Primary and Tertiary RABs In Vivo

To study synergistic interactions between RABs, genome scale metabolic models were used to evaluate the benefit of co-culture for various species (Methods). Overall, RABs were observed to derive greater metabolic support from each other than from other non-RAB species (Wilcoxon p-value<0.001). In particular, tertiary RABs such as B. adolescentis, Ruminococcus bromii and Alistipes shahii could derive metabolic benefits from several other species, including the primary RAB B. thetaiotamicron (FIG. 11, Supplementary Data 7). For investigating potential synergies in vivo and cause-effect relationships, a physiologically relevant mouse model of microbiome recovery was used after antibiotic treatment. Specifically, conventional healthy mice (C57BL/6J, normal gut development, mucin production) were given antibiotics for 5 days before being randomly allocated to four different groups to study treatment effects in a case-control setting: oral gavage with (a) the primary RAB species B. thetaiotamicron (Bt), (b) tertiary RAB species B. adolescentis (Ba), (c) combination of B. thetaiotamicron and B. adolescentis (Bt+Ba), and (d) PBS media (Vehicle; Methods). Recovery was then monitored over a period of 22 days by collecting stool samples every three days and analyzing the microbiome with shotgun metagenomic sequencing (9 timepoints and 2-6 cages per group with 2 mice per cage, Methods; FIG. 4a).

As expected, all treatment groups exhibited a >3-log reduction in microbial biomass after antibiotic treatment (Methods; FIG. 4b). Starting from 1 day after gavage (day 7), and more noticeably at 4 days after gavage (day 10), the Bt and Bt+Ba groups exhibited significantly enhanced biomass recovery (>100×; excluding gavaged species) compared to the PBS and Ba groups (FIG. 4b; FIG. 12a; qPCR verification in Methods). While the Bt and Bt+Ba groups converge to their microbial biomass at pre-antibiotic levels by day 10, the PBS and Ba groups continued to have lower biomass than pre-antibiotic levels at day 22. Enhanced recovery was also associated with successful colonization, confirmed based on comparisons with metagenomic data from a control gavage (Bacillus spp, FIG. 13). Interestingly, the Bt+Ba group was distinct from other treatment groups in recovering higher microbiome diversity at day 19 and 22 (FIG. 4c). This was also accompanied by reconstruction of a community that was more similar to the pre-antibiotic microbiome at day 22 in the Bt+Ba vs the Bt group (FIG. 12b). These results highlight that while Bt gavage and colonization was sufficient for biomass recovery and Ba gavage alone was not, the combination of Bt and Ba promotes biomass and diversity recovery in a synergistic fashion. As observed in the human cohorts, an enrichment of mucin as well as dietary carbohydrate degradation pathways (but not peptidoglycan degradation, as control) was associated with the recovery process in the Bt and Bt+Ba groups (FIG. 4d, e, f).

### DISCUSSION

Cross-cohort analysis is a powerful way to account for confounding effects within individual studies, enabling the identification of consistent associations with microbiome recovery despite variations in cohort characteristics such as antibiotics used and patient demographics. The bacterial species and functions identified in this study provide a data-driven view of how shared microbial factors contribute to gut microbiome recovery in diverse human cohorts around the world, highlighting the value of data-sharing and re-analysis. These findings emphasize the central role of enabling energy harvest from diet, and the ability to colonize the host by degrading mucins in the keystone species that underpin ecological recovery (primary RABs), connecting recovery of key microbiome functions to ecological recovery of biomass and diversity. Additional factors such as antibiotic resistance likely contribute to this process in a time and context-dependent manner. As environmental factors strongly influence the gut microbiome, the specific keystone species that are important for an individual could further vary with host and dietary factors. The analytical approaches used here could uncover these in larger cohorts, helping to train antibiotic and environment-specific machine learning models to predict microbiome recovery. Such models would have clinical utility, especially for at-risk elderly or cancer patients, to guide targeted intervention strategies mitigating the impact of antibiotics on the gut microbiome.

Consistent with the emerging understanding of how diet modulates the gut microbiome, an additional perspective that emerges from this study is the potential to promote RABs and microbiome recovery via prebiotic effects, especially since few RABs are available as probiotics. Many of the identified RABs are specialist carbohydrate fermenters (e.g. pectin) and a high fiber/low fat diet could aid in selecting and expanding them. For example, in a study on how gut microbiota differ in twins discordant for obesity, Ridaura et al identified 3 RABs (B. uniformis, B. thetaiotaomicron and A. putredinis) as being transplantable features of a “lean microbiome”, but transplantation was dependent on a high fiber diet. Similarly, pectin supplementation can promote species from the Bacteroidetes phylum with associated improvement in gut barrier function, as well as more stable fecal microbiota transplantation. Finally, different oligosaccharides can promote the growth of several butyrate producing RABs (Table 2), serving as an avenue to contribute to microbiome recovery by reducing host inflammation and increasing mucin production.

In general, ecological theory has suggested that ecosystem recovery is a complex, multi-step process that is determined by interactions between many species. Observations in the human gut microbiome are in agreement with this model, with the identification of multiple recovery-associated species, the potential for synergistic interactions and microbial cross-feeding, and a conceptual model for how this promotes ecological recovery in the gut. Results from the mouse experiments demonstrate that individual RABs likely have distinct functions, but can work in a synergistic fashion to recover microbial biomass and diversity. As these observations were made in conventional mice with normal physiology (versus germ-free mice), and in a case-control setting where single species gavages (Bt and Ba groups) serve as ideal controls for the combination (Bt+Ba), they highlight the robust role that microbial functions play in the recovery process across species. While investigating all RAB combinations in vivo might be infeasible, systematic investigation of the top predicted metabolic interactions between RABs (e.g. between F. prausnitzii and A. shahii) through in vitro co-cultures could be the next step to unravel the combinatorial interactions among RABs driving microbiome recovery in vivo. Metabolic modeling could, in particular, help further explore the contributions of different carbohydrate degradation genes and processes to microbiome recovery, especially for many anaerobic bacteria that are hard to culture or genetically modify. Further clinical studies incorporating detailed dietary information or with a controlled diet are also needed to evaluate the role of diet and its interaction with RABs and CAZymes in microbiome recovery.

The microbial ‘food-web’ in this study as determined by data-mining techniques is conceptually a valuable resource for organizing an understanding of how microbes interact and assemble in the human gut. Using a large database of human gut microbiome profiles enables the determination of microbial assemblages that are feasible and the dependency relationships that they suggest. These can then help interpret longitudinal studies of recovery and infer the interactions between species that play a role. While current work of the inventors highlights that introduction of primary species such as B. thetaiotamicron is necessary for biomass recovery, in comparison to common probiotics such as B. adolescentis, synergistic combinations can be more beneficial for robust recovery of a diverse gut microbial ecosystem. Similar interactions could also play a critical role in recovery from other microbiome perturbations, and thus a broader understanding of the microbial food-web could set the stage for rational design of pre- and probiotic formulations that promote functional and ecological resilience in gut microbiota.

Whilst there has been described in the foregoing description preferred embodiments of the present invention, it will be understood by those skilled in the technology concerned that many variations or modifications in details of design or construction may be made without departing from the present invention.

1. A method of treating or decreasing gut microbiome dysbiosis induced by a prior antibiotic treatment, the method comprising administering to a subject an effective amount of a composition comprising at least one of or any combination of microorganisms selected from the group consisting of: Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

2. The method according to claim 1, wherein the method comprises administering to a subject an effective amount of a composition comprising: Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

3. The method according to claim 1, wherein the method comprises administering to a subject an effective amount of a composition comprising Bacteroides thetaiotaomicron and Bifidobacterium adolescentis.

4. A synthetic composition for treating or decreasing gut microbiome dysbiosis induced by a prior antibiotic treatment, the composition comprising at least one of or a combination of a microorganisms selected from the group consisting of Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

5. The composition according to claim 4, wherein the composition comprises Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

6. The composition according to claim 4, wherein the composition comprises Bacteroides thetaiotaomicron and Bifidobacterium adolescentis.

7. The composition according to claim 4, wherein the composition is a probiotic, food product or a pharmaceutical composition.

8. The composition according to claim 7, wherein the pharmaceutical composition is formulated for oral administration.

9. The composition according to claim 7, wherein the microorganism is lyophilised, pulverised and powdered.

10. The composition according to claim 7, wherein the microorganism is a liquid culture.

11. The composition according to claim 7, further comprising a coating, optionally wherein the coating is an enteric coating.

12. The composition according to claim 11, wherein the coating is made of a material comprising at least one of a saccharide, a polysaccharide, and a glycoprotein extracted from at least one of a plant, a fungus, and a microbe, optionally wherein the at least one of a saccharide, a polysaccharide, and a glycoprotein includes one or more of corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum.

13. The composition according to claim 7, wherein the pharmaceutical composition is formulated with a germinant.

14. The composition according to claim 7, wherein the composition is formulated in a dosage form at least about 1×10⁴ colony forming units of bacteria.

15. A method for predicting the likelihood of antibiotics-induced microbiome dysbiosis recovery in a subject, the method comprising:
(a) determining a gut microbiome signature of the subject by determining an amount of, or presence or absence of, each microorganism in a group of microorganisms present in a sample obtained from the subject; and
(b) applying a prediction model to assess the gut microbiome signature with respect to a gut profile representative of good gut health to obtain a likelihood of antibiotics-induced microbiome dysbiosis recovery in the subject,
wherein the group of microorganisms comprises Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile, and
wherein the prediction model is trained using a dataset of microbiome profiles of a plurality of subjects who have recovered from antibiotics-induced microbiome dysbiosis, and a plurality of subject who have not recovered from antibiotics-induced microbiome dysbiosis.

16. The method according to claim 15, wherein the prediction model comprises a machine learning probability model.

17. The method according to claim 16, wherein the prediction model comprises a random forest classification model, or a linear discriminant analysis model, or a sparse logistic regression model, or a conditional inference tree model.

18. The method according to claim 15, wherein the sample is a faecal sample obtained from the subject.

19. The method according to claim 15, further comprising administering to the subject an effective amount of a composition comprising at least one of or any combination of microorganisms selected from the group consisting of: Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

20. A method for reducing antibiotics-induced gut microbiome dysbiosis in a subject, the method comprising:
(a) determining a gut microbiome signature of the subject by determining an amount of, or presence or absence of, each microorganism in a group of microorganisms present in a sample obtained from the patient; and
(b) administering to the subject a therapeutically effective amount of an agent which up-regulates at least one microbe which is down-regulated during a prior antibiotic treatment or administering to the subject a therapeutically effective amount of an agent which down-regulates a microbe which is up-regulated during a prior antibiotic treatment, thereby reducing antibiotics-induced gut microbiome perturbations in a subject,
wherein the class of microbes comprises Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

21. The method according to claim 20, wherein the agent is a probiotic and/or a prebiotic.

22. The method according to claim 21, wherein the probiotic is a bacterial population comprises Alistipes putredinis, Alistipes shahii, Bacteroides caccae, Bacteroides coprocola, Bacteroides eggerthii, Bacteroides intestinalis, Bacteroides stercoris, Bacteroides thetaiotaomicron, Bacteroides uniformis, Bifidobacterium adolescentis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

23. A method of determining the effect of a perturbation on a gut microbial community, the method comprising applying the perturbation to a cultured collection of a gut microbial community and determining the difference in the community before and after the application of the perturbation, wherein the difference in the cultured collection represents the effect of the perturbation on the original gut microbial community, wherein the gut microbial community comprises Bifidobacterium adolescentis, Bacteroides thetaiotaomicron, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile.

24. The method according to claim 23, wherein the perturbation is a diet related perturbation, an environmental perturbation, a genetic perturbation or a pharmaceutical perturbation.

25. A computer readable storage medium comprising computer readable instructions operable when executed by a computer to determine the likelihood of antibiotics-induced gut microbiome recovery in a subject, the computer readable instructions configured to perform a method of claim 15.

26. An apparatus or system comprising:
(a) a receiving unit configured to receive a dataset of values representing a gut microbiome signature of a subject by determining an amount of, or presence or absence of, each microorganism in a group of microorganisms present in a sample obtained from the subject; and
(b) a processor configured to process a prediction model to assess the gut microbiome signature with respect to a gut profile representative of good gut health to obtain a likelihood of antibiotics-induced microbiome dysbiosis recovery in the subject,
wherein the group of microorganisms comprises Bacteroides thetaiotaomicron, Bifidobacterium adolescentis, Alistipes putredinis, Alistipes shahii, Bacteroides caccae, Bacteroides coprocola, Bacteroides eggerthii, Bacteroides intestinalis, Bacteroides stercoris, Bacteroides uniformis, Bifidobacterium bifidum, Bifidobacterium longum, Coprococcus catus, Desulfovibrio piger, Faecalibacterium prausnitzii, Parabacteroides distasonis, Parabacteroides johnsonii, Roseburia inulinivorans, Ruminococcus bromii, Ruminococcus torques, and Subdoligranulum variabile, and
wherein the prediction model is trained using a dataset of microbiome profiles of a plurality of subjects who have recovered from antibiotics-induced microbiome dysbiosis, and a plurality of subjects who have not recovered from antibiotics-induced microbiome dysbiosis.
